SEQUENCE LISTING (20 sequences)

SEQ ID NO: 1 - DNA, 197 bp, Rattus norvegicus
gggacaaaactttataactacaagcacttaagcctcaaaattcttgactttttctttaatgactatagtataaccctcagttggtcacatgtctacacattatttccagttgataacaagtagcggtgttttccatatgtaattcagatctgaacttaatggcaataaatggtttaaatatttgcgaaaaaaaaaaa

SEQ ID NO: 2 - DNA, 167 bp, Rattus norvegicus
caggtcacggaagccagtcatgccggagacaccggcttctgggaagccgcccaggtctcattcctccctgctgtttggaggcagcatctcctctttttatggagggcccgtccttttttcttacaaattcttcaataaagacacattcttgaggcgaaaaaaaaaaa

SEQ ID NO: 3 - DNA, 901 bp, Rattus norvegicus
gctggccgggtcgaccctggtgtcatccgtttaggaagcggcttcaccgccaacagcacggccatggctggagctctggtgcgcaaagcagcggactatgtccggagcaaggacttccgggactatctcatgagtacgcacttctggggcccagttgccaactggggtctccccattgctgctatcaatgacatgaagaaatctccagagattatcagtgggcggatgactttcgccctctgttgctattctctgacattcatgagatttgcctacaaggtacaaccccgaaactggcttctgtttgcgtgccatgtgacaaacgaagtcgctcagctcattcagggaggacgacttatcaactacgagatgagtaagcggccatctgcctagcagtgcaaggaccagctcttgaaagggacagtgctccagccactgttgcggccacagatcacgtcagcatgaatagtcgtgctgaggggaaaacacggaagactatctttaatgaccatgccaacattattgaatagccaagaatccccaaaccaactctcggctgccttatcaatgctaaactttatttgtcttcatcaggagtagttcaaaatatgcagctaatttaataattttgaatgatgttatctatagcaatctgtagtaatatgtatattatctattgggatttgtgtaataaaaaatctaagggaacaaaactttataactacaagcacttaagtcctcaaaattcttgactttttctttaatgactatagtataaccctcagttggtcacatgtctacacataatttccagtgataacaagtagcggtgttttccatatgtaattcagatctgaacttaatggcaataaatggtttaaatatttgcgaaaaaaaaaaa

SEQ ID NO: 4 - protein, 109 aa, Rattus norvegicus
MAGALVRKAADYVRSKDFRDYLMSTHFWGPVANWGLPIAAINDMKKSPEIISGRMTFALCCYSLTFMRFAYKVQPRNWLLFACHVTNEVAQLIQGGRLINYEMSKRPSA

SEQ ID NO: 5 - protein, 109 aa, Mus musculus
MAGALVRKAADYVRSKDFRDYLMSTHFWGPVANWGLPIAAINDMKKSPEIISGRMTFALCCYSQTFMRFAYKVQPRNWLLFACHVTNEVAQLIQGGRLINYEMSKRPSA

SEQ ID NO: 6 - protein, 102 aa, Homo sapiens
MAGALVRKAADYVRSKDFRDYLMSTHFWGPVANWGLPIAAINDMKKSPEIISGRMTFALCCYSLTFMRFAYKVQPRNWLLFACHATNEVAQLIQGGRLIKHE

SEQ ID NO: 7 - DNA, 988 bp, Homo sapiens
gtcgtgaggcgggccttcgggctggctcgccgtcggctgccggggggttggcctgggtgtcattggctctgggaagcggcagcagaggcagggaccactcggggtctggtgtcggcacagccatggcgggcgcgttggtgcggaaagcggcggactatgtccgaagcaaggatttccgggactacctcatgagtacgcacttctggggcccagtagccaactggggtcttcccattgctgccatcaatgatatgaaaaagtctccagagattatcagtgggcggatgacatttgccctctgttgctattctttgacattcatgagatttgcctacaaggtacagcctcggaactggcttctgtttgcatgccacgcaacaaatgaagtagcccagctcatccagggagggcggcttatcaaacacgagatgactaaaacggcatctgcataacaatgggaaaaggaagaacaaggtcttgaagggacagcattgccagctgctgctgagtcacagatttcattataaatagcctccctaaggaaaatacactgaatgctatttttactaaccattctatttttatagaaatagctgagagtttctaaaccaactctctgctgccttacaagtattaaatattttacttctttccataaagagtagctcaaaatatgcaattaatttaataatttctgatgatgttttatctgcagtaatatgtatatcatctattagaatttacttaatgaaaaactgaagagaacaaaatttgtaaccactagcacttaagtactcctgattcttaacattgtctttaatgaccacaagacaaccaacagctggccacgtacttaaaattttgtccccactgtttaaaaatgttacctgtgtatttccatgcagtgtatatattgagatgctgtaacttaatggcaataaatgatttaaatatttgttaaaaaaaaaaaaaaaaaaaaaaaaaaaaa

SEQ ID NO: 8 - DNA, 873 bp, Mus musculus
ggtgtcatctgtctaggtagcggcttcaccgccaacggcacggccatggctggagcgctggtgcgcaaagcggcggactatgtccggagcaaggacttccgggactatctcatgagtacgcacttctggggcccagttgccaactggggtctccccattgctgctatcaatgacatgaagaaatctccagagattatcagtgggcggatgactttcgccctctgttgctattctctgacattcatgagatttgcctacaaggtacaacctcgaaactggcttttgtttgcatgccatgtaacaaacgaagtagctcagctcattcagggaggacgacttatcaactacgagatgagtaagcggccatctgcatagcggtacaaggaccagctcttgaaagagacagtgctccagccactgctgcagccacagatcatgtcagcatgagtagtcgtgctgaagggaaaacacagaatgctatcttaatgaccatgccaacattattgaatagccgagagtccctaaacccactctctgctgccttatcaatgctaaaccttatttgtcttcatcaagagtagttcaaaatatgcaactaatttaataattttgaatgatggttttatctatagcaatctgtagtaatatgtatattatctattgggatttgtgtaataaaaaatctaagggaacaaaattttataactacaagcacttaagtactcaaaattcttgactttttctttaatgacaatagtaaaccctcagttggtcacatgtctacacataatttccagtgataacaagtatcggtgttttccatatgtaactcagatctgtaacttaatggcaataaatggtttaaatatttgc

SEQ ID NO: 9 - DNA, 549 bp, Mus musculus
cggcacagccatggcgggcgcgttggtgcggaaagcggcggactatgtccgaagcaaggatttccgggactacctcatgagtacgcacttctggggcccagtagccaactggggtcttcccattgctgccatcaatgatatgaaaaagtctccagagattatcagtgggcggatgacatttgccctctgttgctattctttgacattcatgagatttgcctacaaggtacagcctcggaactggcttctgtttgcatgccacgcaacaaatgaagtagcccagctcatccagggagggcggcttatcaaacacgagatgactgtaacttaatggcaataaatgatttaaatatttgaagagtagctcaaaatatgcaattaatttaataatttatctgcagtaatatgtatatcatctattagaatttacttaatgaaaaactgaagagaacaaaatttgtaaccactagcacttaagtactcctgattcttaacattgtctttaatgacaatagctgagagtttctaaaccaactctctgctgcctta

SEQ ID NO: 10 - DNA, 10 bp, Artificial Sequence
  5′ random arbitrary primer
caagcgaggt

SEQ ID NO: 11 - DNA, 10 bp, Artificial Sequence
  5′ random arbitrary primer
cagtgagctg

SEQ ID NO: 12 - DNA, 10 bp, Artificial Sequence
  5′ random arbitrary primer
gtcacggaag

SEQ ID NO: 13 - DNA, 27 bp, Artificial Sequence
  PCR primer AP1
ccatcctaatacgactcactatagggc

SEQ ID NO: 14 - DNA, 24 bp, Artificial Sequence
  PCR primer corresponding to SEQ ID NO 1
agccgagagttggtttggggattc

SEQ ID NO: 15 - DNA, 22 bp, Artificial Sequence
  Primer I  (ARBP cDNA  5′ primer)
gggatccaacagcacggccatg

SEQ ID NO: 16 - DNA, 26 bp, Artificial Sequence
  primer II  (ARBP cDNA 3′ primer )
ggaattcattgataaggcagccgaga

SEQ ID NO: 17 - DNA, 20 bp, Artificial Sequence
  GAPDH sense primer
tgctggtgctgagtatgtcg

SEQ ID NO: 18 - DNA, 20 bp, Artificial Sequence
  GAPDH anti-sense primer
gcatgtcagatccacaacgg

SEQ ID NO: 19 - protein, 14 aa, Artificial Sequence
  polyclonal antibody against a C-terminal peptide sequence of ARBP
GRLINYEMSKRPSA

SEQ ID NO: 20 - DNA, 550 bp, Homo sapiens
cggcacggccatggctggagcgctggtgcgcaaagcggcggactatgtccggagcaaggacttccgggactatctcatgagtacgcacttctggggcccagttgccaactggggtctccccattgctgctatcaatgacatgaagaaatctccagagattatcagtgggcggatgactttcgccctctgttgctattctctgacattcatgagatttgcctacaaggtacaacctcgaaactggcttttgtttgcatgccatgtaacaaacgaagtagctcagctcattcagggaggacgacttatcaactacgagatgactgtaacttaatggcaataaatggtttaaatatttgaagagtagttcaaaatatgcaactaatttaataatttatctgtagtaatatgtatattatctattgggatttgtgtaataaaaaatctaagggaacaaaattttataactacaagcacttaagtactcaaaattcttgactttttctttaatgacaatagccgagagtccctaaacccactctctgctgcctta

(19) United States
(12) Patent Application Publication
(10) Pub. No.: US 2004/0254111 A1
(43) Pub. Date: Dec. 16, 2004

(54) Regulator of apoptosis and cell proliferation

(76) Inventors: Huang Bing-Ren, Beijing (CN); Zhou Xin-Fu, Seacombe Heights (AU)

Correspondence Address: BIRCH STEWART KOLASCH & BIRCH, PO BOX 747, FALLS CHURCH, VA, 22040-0747, US

(21) Appl. No.: 10/791,860

(22) Filed: Mar. 4, 2004

(30) Foreign Application Priority Data
Mar. 6, 2003 (AU) 2003901010

(51) Int. Cl. A61K 38/17

(52) U.S. Cl. 514/12

(57) ABSTRACT

This invention relates to a method of regulating apoptosis and/or proliferation in a cell, the method including the step of altering the expression and/or function in the cell of polypeptides based on Apoptosis Related Basic Protein.

### FIELD OF THE INVENTION

The present invention relates to methods of regulating apoptosis and cell proliferation, and also to agents that regulate apoptosis and cell proliferation.

### BACKGROUND OF THE INVENTION

Apoptosis is the process by which cells undergo programmed cell death. Apoptosis is a genetically controlled process in which unneeded or damaged cells can be eliminated without causing tissue destruction and an inflammatory response. Such programmed cell death is a fundamental and essential process in the development and tissue homeostasis of multicellular organisms. For example, neuronal apoptosis plays an indispensable role in the development of the nervous system. Approximately half of all neurons produced during neurogenesis die apoptotically before maturation of the nervous system.

During apoptosis, the chromatin condenses and the nucleus breaks up into small pieces. The chromosomal DNA is usually fragmented as a result of cleavage between nucleosomes. Finally, the cell shrinks and breaks up into membrane-enclosed fragments, called apoptotic bodies, which are readily recognized and phagocytosed by both macrophages and neighboring cells.

A variety of different molecules play a role in regulating apoptosis. Although a number of these molecules involved in regulating apoptosis have been identified, the detailed molecular events involved in regulating the process still remain to be elucidated.

The processes and mechanisms regulating apoptosis are highly conserved in all organisms. Dysregulation of apoptosis has also recently been recognized as a significant factor in the pathogenesis of many human diseases and conditions.

As apoptosis plays an important role in development and the pathogenesis of many diseases and conditions, there is a need to identify new methods and agents that are able to regulating the process. The present invention relates to the identification of a protein involved in the regulation of apoptosis and cell proliferation, and to methods and agents for regulating apoptosis and cell proliferation based on regulating the expression and function of this protein.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY OF THE INVENTION

The present invention provides a method of regulating apoptosis in a cell, the method including the step of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% identical to an amino acid sequence of SEQ ID No. 4, or a biologically active fragment thereof.

The present invention also provides a method of regulating apoptosis in a cell, the method including the step of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80% identical to a nucleotide sequence complementary to SEQ ID No. 3, or RNA equivalent thereof;

(e) a nucleotide sequence encoding a biologically active fragment of a polypeptide having an amino acid sequence of SEQ ID No.4;

(f) a nucleotide sequence encoding an antisense nucleic acid that reduces the expression in the cell of a polypeptide including an amino acid sequence of SEQ ID No.4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

The present invention further provides a method of regulating proliferation of a cell, the method including the step of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4 and

(c) an amino acid sequence at least 90% identical to an amino acid sequence of SEQ ID No. 4, or a biologically active fragment thereof.

The present invention also provides a method of regulating proliferation of a cell, the method including the step of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80% identical to a nucleotide sequence complementary to SEQ ID No. 3, or RNA equivalent thereof;

(e) a nucleotide sequence encoding a biologically active fragment of a polypeptide having an amino acid sequence of SEQ ID No. 4;

(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

The present invention also provides an agent for regulating cell apoptosis and/or proliferation, wherein the administration of an effective amount of the agent to a cell alters the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% identical to an amino acid sequence of SEQ ID No. 4, or a biologically active fragment thereof.

The present invention also provides a composition for regulating apoptosis and/or cell proliferation, the composition including a polypeptide including an amino acid sequence selected from the group consisting of:

(a) an amino acid sequence of SEQ ID No.4;

(b) an amino acid sequence of a biologically active fragment of a polypeptide having an amino acid sequence of SEQ ID No.4; and

(c) amino acid sequence at least 90% identical to an amino acid sequence of SEQ ID No.4, or a biologically active fragment thereof.

The present invention also provides an antibody raised against a polypeptide including an amino acid sequence selected from the group consisting of:

(a) an amino acid sequence of SEQ ID No.4;

(b) an amino acid sequence encoding a biologically active fragment of a polypeptide having an amino acid sequence of SEQ ID No.4;

(c) an amino acid sequence at least 90% identical to an amino acid sequence of SEQ ID No.4, or a biologically active fragment thereof; and

(d) an amino acid sequence encoding an immunogenic fragment of a polypeptide having an amino acid sequence of SEQ ID No.4.

The present invention also provides an antibody that detects a polypeptide including an amino acid sequence selected from the group consisting of:

(a) an amino acid sequence of SEQ ID No. 4;

(b) an amino acid sequence encoding a biologically active fragment of a polypeptide having an amino acid sequence of SEQ ID No. 4;

(c) an amino acid sequence at least 90% identical to an amino acid sequence of SEQ ID No. 4, or a biologically active fragment thereof; and

(d) an amino acid sequence encoding an immunogenic fragment of a polypeptide having an amino acid sequence of SEQ ID No.4.

The present invention further provides a vector including a nucleic acid sequence selected from the group consisting of:

(a) a nucleic acid having a nucleotide sequence of SEQ ID No. 3;

(b) a nucleic acid having a nucleotide sequence at least 80% identical to a nucleotide sequence of SEQ ID No. 3;

(c) a nucleic acid having a nucleotide sequence complementary to SEQ ID No. 3;

(d) a nucleic acid having a nucleotide sequence at least 80% identical to a nucleotide sequence complementary to SEQ ID No. 3;

(e) a nucleic acid sequence encoding a biologically active fragment of a polypeptide having an amino acid sequence of SEQ ID No. 4;

(f) a nucleic acid encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleic acid encoding a ribozyme that cleaves a mRNA encoding an amino acid of SEQ ID No. 4 or an amino acid sequence 90% identical to an amino acid sequence of SEQ ID No. 4.

The present invention also provides a detectably labelled nucleotide probe, the probe including a nucleotide sequence that hybridises under stringent conditions to a nucleic acid sequence having a nucleotide sequence of SEQ ID No. 3 or a nucleic acid having a nucleotide sequence complementary to SEQ ID No. 3.

The present invention also provides a method of detecting a nucleic acid encoding an amino acid sequence of SEQ ID No. 4 or an amino acid sequence at least 90% identical to an amino acid sequence of SEQ ID No. 4, the method including the step of hybridizing to the nucleic acid a detectably labelled nucleotide probe that hybridizes to the nucleic acid under stringent conditions.

The present invention also provides a method of detecting a nucleic acid encoding an amino acid sequence of SEQ ID No. 4 or an amino acid sequence at least 90% identical to an amino acid sequence of SEQ ID No. 4, the method including the step of hybridizing to the nucleic acid a first nucleic acid that specifically amplifies the nucleic acid in conjunction with a second nucleic acid.

The present invention also provides an antisense nucleic acid that reduces the expression in a cell of a polypeptide including an amino acid sequence of SEQ ID No. 4 or an amino acid sequence at least 90% identical to an amino acid sequence of SEQ ID No. 4.

The present invention further provides a synthetic ribozyme that cleaves a mRNA encoding an amino acid sequence of SEQ ID No. 4 or an amino acid sequence at least 90% identical to an amino acid sequence of SEQ ID No. 4.

The present invention arises out of studies into genes expressed in cells undergoing apoptosis. In particular, differential display has led to the identification of a protein, designated ARBP (for Apoptosis Related Basic Protein). ARBP is expressed in a variety of cell types. Altering the expression of the ARBP protein by antisense RNA expression can regulate apoptosis and cell proliferation. Antisense experiments indicate that ARBP may function in cells as a regulator of the cell cycle, acting normally to suppress mitosis.

Various terms that will be used throughout the specification have meanings that will be well understood by a skilled addressee. However, for ease of reference, some of these terms will now be defined.

The term “nucleic acid” as used throughout the specification is to be understood to mean to any oligonucleotide or polynucleotide. The nucleic acid may be DNA or RNA and may be single stranded or double stranded. The nucleic acid may be any type of nucleic acid, including a nucleic acid of genomic origin, cDNA origin (ie derived from a mRNA), derived from a virus, or of synthetic origin.

The term “polypeptide” as used throughout the specification is to be understood to mean two or more amino acids joined by peptide bonds. Similarly, the term “amino acid sequence” refers to an oligopeptide, peptide, polypeptide, or protein sequence, and fragments or portions thereof, and to naturally occurring, recombinant, mutated or synthetic polypeptides.

The term “variant” as used throughout the specification is to be understood to mean an amino acid sequence that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids.

The term “biologically active fragment” as used throughout the specification with reference to a polypeptide is to be understood to mean a polypeptide having similar structural, regulatory, or biochemical functions as that of the full size polypeptide. For example, a biologically active fragment may be an amino or carboxy terminal deletion of a polypeptide, an internal deletion of a polypeptide, or any combination of such deletions. A biologically active fragment will also include any such deletions fused to one or more additional amino acids.

The term “amplification” or variants thereof as used throughout the specification is to be understood to mean the production of additional copies of a nucleic acid sequence. For example, amplification may be achieved using polymerase chain reaction (PCR) technologies, essentially as described in Dieffenbach, C. W. and G. S. Dveksler (1995) PCR Primer, a Laboratory Manual, Cold Spring Harbor Press, Plainview, N.Y.

The term “hybridization” or variants thereof as used throughout the specification is to be understood to mean any process by which a strand of nucleic acid binds with a complementary strand through base pairing. Hybridization may occur in solution or between one nucleic acid sequence present in solution and another nucleic acid sequence immobilized on a solid support (e.g., membranes, filters, chips etc).

The term “stringent conditions” as used throughout the specification is to be understood to mean the conditions that allow complementary nucleic acids to bind to each other within a range from at or near the Tm (Tm is the melting temperature) to about 20° C. below Tm. Factors such as the length of the complementary regions, type and composition of the nucleic acids (DNA, RNA, base composition), and the concentration of the salts and other components (e.g. the presence or absence of formamide, dextran sulfate and/or polyethylene glycol) must all be considered, essentially as described in in Current Protocols in Molecular Biology, John Wiley & Sons, N.Y. (1989).

The term “antisense nucleic acid” as used throughout the specification is to be understood to mean a nucleotide sequence which is fully or partially complementary to a specific target DNA or RNA sequence. Antisense nucleic acids may be produced by a suitable method known in the art, including expression from a recombinant nucleic acid or chemical synthesis. Once introduced into a cell, the antisense nucleic acid combines with other nucleic acid sequences present in the cell to form duplexes and reduce the expression of the polypeptide of interest.

The term “immunogenic fragment” as used throughout the specification is to be understood to mean a portion of a polypeptide that is able to be recognised by a particular antibody. When a polypeptide is used to immunize a host animal, numerous regions of the polypeptide may induce the production of antibodies which bind specifically to a given region or three-dimensional structure on the polypeptide. An immunogenic fragment will include one or more of these regions that may bind specifically with the antibody.

The term “antibody” as used throughout the specification is to be understood to mean monoclonal or polyclonal antibodies, and fragments of antibody molecules, such as Fab, F(ab′)2, and Fv, which are capable of binding an epitopic determinant. In this regard, the term will be understood to include within its scope a humanized antibody, in which amino acids have been replaced in the non-antigen binding regions in order to more closely resemble a human antibody, while still retaining the original binding ability.

### BRIEF DESCRIPTION OF THE FIGURES

FIG. 1 shows the results of DNA fragmentation analysis of R2L1 cells upon serum deprivation. Lane 1 shows the extent of DNA fragmentation in R2L1 cells upon serum deprivation for 24 hours. Lane 2 shows the extent of DNA fragmentation in R2L1 cells when cultured in medium containing serum for 24 hours. Lane 3 shows λ DNA cut with HindIII.

FIG. 2 shows the results of flow cytometric analysis of R2L1 cells upon serum deprivation.

FIG. 3 shows the results of the analysis of differentially expressed genes by the differential display technique. Lane 1, 2 and 3 are amplified gene fragments from serum-containing R2L1 cells, serum-deprived R2L1 and serum-deprived R2P cells, respectively. Arrows indicated two differentially expressed bands.

FIG. 4 shows the expression level of mRNA encoding SEQ ID No. 1, SEQ ID No. 2 and β-actin in different cells by Northern blotting. Lane 1 is RNA from serum-containing cultured R2L1 cells. Lane 2 is RNA from R2L1 cells serum-deprived R2L1 cells. Lane 3 is RNA from R2P cells serum-deprived for 6 hours.

FIG. 5 shows the nucleotide sequence of the full-length ARBP cDNA (SEQ ID No. 3) and the predicted amino acid sequence of the encoded protein (SEQ ID No. 4).

FIG. 6 shows the alignment of the predicted amino acid sequences of the rat ARBP protein with the predicted protein encoded by the nucleic acid of the mouse homologue (GenBank NM_{—}018819) and the predicted protein encoded by the nucleic acid of the human homologue (GenBank NM_{—}016098) using the CLUSTLAW algorithm.

FIG. 7 shows the homology between SEQ ID No. 3 and the nucleotide sequence of the predicted human homologue (GenBank NM_{—}016098) using the BLAST algorithm.

FIG. 8 shows the homology between SEQ ID No. 3 and the nucleotide sequence of the predicted mouse homologue (GenBank NM_{—}018819) using the BLAST algorithm.

FIG. 9 shows the homology between predicted ARBP mouse homologue (GenBank NM_{—}018819) and the human homologue (GenBank NM_{—}016098) using the BLAST algorithm.

FIG. 10 shows detection of changes in ARBP mRNA in dorsal root ganglia (DRG) of adult rats 3 days after sciatic nerve transection by a semi-quantitative method using GAPDH as internal control. ARBP mRNA in the lesioned DRG is increased as compared with contralateral intact DRG. IP: Ipsilateral lesioned side; CL: Contralateral lesioned side.

FIG. 11 shows a Western blot of detection of ARBP in superior cervical ganglia (SCG), dorsal root ganglia (DRG), trigeminal ganglia (Tri. G) and olfactory bulb (Olf. B) of adult rat. Two bands were found at 12.5 and 9.5 kD.

FIG. 12 shows immunohistochemistry analysis of ARBP expression.

FIG. 13 shows immunolocalization of ARBP in the central and peripheral nervous systems and in peripheral tissues of the adult rat.

FIG. 14 shows growth curves of R2L1 stably transfected with sense and antisense ARBP expression constructs.

FIG. 15 shows the percentage survival of cells transfected with sense and anti-sense ARBP expression constructs after deprivation of serum for 24h by flow cytometry analysis.

### GENERAL DESCRIPTION OF THE INVENTION

As mentioned above, in one form the present invention provides a method of regulating apoptosis in a cell, the method including the step of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% homologous to an amino acid sequence of SEQ ID No. 4, or a biologically active fragment thereof.

The method of this form of the present invention provides the ability to regulate apoptosis by altering the expression and/or function in a cell of a polypeptide including an amino acid sequence selected from the group consisting of: (i) an amino acid sequence of SEQ ID No. 4; (ii) an amino acid sequence of a biologically active fragment of a polypeptide having an amino acid sequence of SEQ ID No. 4; and (iii) an amino sequence at least 90% identical to an amino acid sequence of SEQ ID No. 4, or a biologically active fragment thereof.

The cell is any eukaryotic cell that has the capacity to undergo apoptosis. For example, the cell may be a human cell, a mouse cell or a rat cell. An example of a cell type for which apoptosis may be regulated by the method of the present invention is a neuronal cell.

The cell may be a cell present in an entire organism, such as an animal or human, or alternatively may be a cell cultured in vitro, such as a cell grown in tissue culture. For example, the cell may be an isolated cell (such as a human or rat neuronal cell), a cell that has the capacity to undergo apoptosis present in a tissue or organ in an animal or human subject, or a cell that is undergoing apoptosis present in a tissue or organ in an animal or human subject. The cell may also be a cell having dysregulation of apoptosis or a cell associated with a disease or condition involving dysregulation of apoptosis.

Examples of cells for which apoptosis may be regulated include cells in the ventral horn (neurons), adrenal medulla (neurons), adrenal cortex, dorsal root ganglion, trigeminal ganglia, hypoglossal nucleus, peripheral nerve (Schwann cells), caudal ventral lateral medulla, cerebellar deep nuclei, singular cortex, dorsal column, dorsal horn, footpad sweat gland, gut, hippocampus, kidney medulla, liver, lung, lymph nodes, mid brain, salivary gland, skin, spleen, substantia nigra, sweat gland, testes, and thymus.

Preferably the cell is a neuronal cell. More preferably, the neuronal cell is a rat or human neuronal cell. Examples of neuronal cells include neuronal cells present in substantia nigra, dorsal root ganglia, trigeminal ganglia and olfactory bulb, motor neurons and hypoglossal neurons.

Examples of non-neuronal cell types for which apoptosis may be regulated include non-neuronal cells present in neural and brain tissue, intestinal cells including goblet cells, lymph node cells, spleen cells, liver cells, thymic cells, salivary gland cells, pituitary cells, bladder cells, bone cells, breast cells, cervical cells, colorectal cells, kidney cells, laryngeal cells, blood cells, lung cells, lymphatic cells, skin cells, plasma cells, muscle cells, cells of the mouth or throat, ovaries, pancreatic cells, prostate cells, stomach cells, testicular cells, germ cells, thyroid cells, and uterine cells.

Preferably the cell has the capacity to undergo apoptosis mediated by a neurotrophin receptor and/or the binding of a neurotrophin to a receptor. Examples of neurotrophin receptors include TrkA, TrkB, TrkC, and p75NTR. More preferably, the cell has the capacity to undergo apoptosis mediated by p75NTR, or a cell that has the capacity to undergo apoptosis mediated by the binding of a neurotrophin to p75NTR. Examples of neurotrophins that have the capacity to bind to p75NTR include NGF, BDNF, NT-3, NT-4/NT-5 and NT-6.

Preferably, the cell is a neuronal cell that has the capacity to undergo apoptosis mediated by a neurotrophin receptor and/or the binding of a neurotrophin to a receptor.

Accordingly, in a preferred form, the present invention provides a method of regulating apoptosis of a neuronal cell mediated by a neurotrophin receptor and/or binding of a neurotrophin to a receptor, the method including the steps of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% homologous to an amino acid sequence of SEQ ID No. 4, or a biologically active fragment thereof.

The neurotrophin receptor may be a receptor normally expressed endogenously in the cell of interest, or may be a receptor introduced into a cell, for example by way of transient or stable transformation, or by viral infection or transduction, by methods known in the art.

The neurotrophin receptor introduced into the cell may be from the same species or from a different species as the recipient cell, and may consist of the entire receptor or a biologically active fragment or variant of the receptor. For example, the entire human neurotrophin receptor p75NTR may be introduced into a rat neuronal cell.

The neurotrophin may be any neurotrophin that may bind to a receptor present in a cell and cause apoptosis. Preferably, the neurotrophin is NGF, BDNF, NT-3, NT-4/NT-5 and NT-6.

The regulation of apoptosis in the various forms of the present invention is any form of control or change of the initiation or progression of apoptosis in a cell. For example, regulation of apoptosis may involve (i) reducing or promoting the ability of a cell to enter apoptosis; (ii) reducing or promoting the progression of apoptosis in a cell after apoptosis has begun; and/or (iii) reducing or promoting the probability that a particular cell will begin or progress through apoptosis.

In a preferred form, the present invention provides a method of inhibiting apoptosis in a cell, the method including the step of reducing the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% homologous to an amino acid sequence of SEQ ID No. 4, or a biologically active fragment thereof.

The apoptosis in the various forms of the present invention may be any form of apoptosis that occurs in a cell in response to a genetic or external response, or be any form of dysregulated apoptosis. For example, the apoptosis may be induced in response to the presence or absence of a factor, such as the apoptosis that occurs in neuronal cells mediated by a neurotrophin receptor, or the induction of apoptosis that occurs in some cell types upon serum deprivation.

Preferably, the apoptosis is mediated by a neurotrophin receptor and/or the binding of a neurotrophin to a receptor. More preferably, the apoptosis is mediated by one or more of the neurotrophin receptors TrkA, TrkB, TrkC or p75NTR. Most preferably, the apoptosis is mediated by p75NTR.

Examples of diseases mediated by a dysregulation of apoptosis include viral diseases such as AIDS; fulminant hepatitis; neurodegenerative diseases such as Alzheimer's disease, Parkinson's disease; dementia; demyelination diseases; cancers in which dysregulation of apoptosis is associated with development of the cancerous phenotype; amyotrophic lateral sclerosis; retinitis pigmentosa; cerebellar degeneration; myelodysplasis such as aplastic anemia; ischemic diseases such as myocardial infarction and stroke; hepatic diseases such as alcoholic hepatitis, hepatitis B and hepatitis C; joint diseases such as osteoarthritis; and atherosclerosis.

Examples of cancers in which dysregulation of apoptosis is associated with development of the cancerous phenotype include bladder cancer, bone cancer, brain tumours, breast cancer, cervical cancer, colorectal cancer including cancer of the colon, rectum, anus, and appendix, cancer of the esophagus, Hodgkin's disease, kidney cancer, cancer of the larynx, leukemia, liver cancer, lung cancer, lymphoma, melanoma, moles and dysplastic nevi, multiple myeloma, muscular cancer, non-Hodgkin's lymphoma, oral cancer, ovarian cancer, cancer of the pancreas, prostate cancer, skin cancer, stomach cancer, testicular cancer, teratoma, thyroid cancer, cancer of the uterus.

Methods for determining the extent of apoptosis in a cell in the various forms of the present invention may be performed by a suitable method known in the art, including (i) DNA fragmentation assays, for example terminal deoxynucleotidyl transferase-mediated dUTP nick end labeling (TUNEL), which is an in situ method for detecting the 3′-OH ends of DNA exposed during the internucleosomal cleavage that occurs during apoptosis, and which may be performed essentially as described in Hensey C. and Gautier J. (1998). Dev. Biol. 203, 36-48; Veenstra, G J, Peterson-Maduro J, Mathu M T, van der Vliet P C, Destree O H J. (1998). Cell Death Differ 5:774-84.; (ii) detection of morphological changes associated with apoptosis, essentially as described in Compton M M (1992) Cancer Metast Rev 11:105-119, 1992; Wyllie A H (1992) Cancer Metast Rev 11: 95-103; Oltvai Z N, Korsmeyer S J (1994) Cell 79:189-192, 1994; or (iii) use of flow cytometry analysis to detect apoptosis, essentially as described in Ormerod M G, Collins M K L, Rodriguez-Tarduchy G, Robertson D (1992) J Immunol Meth 153:57-66; Jacobs D P, Pipho C (1983) J Immunol Meth 62: 101 -110.

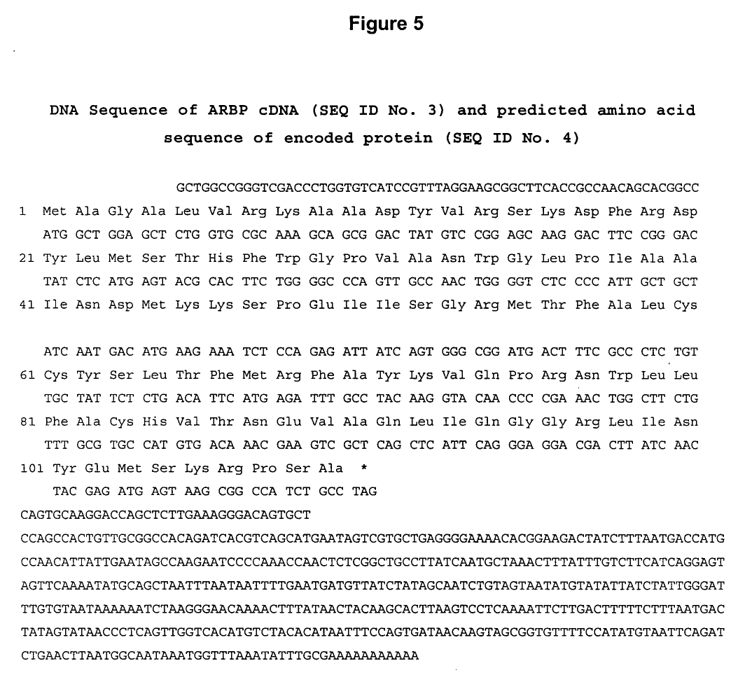

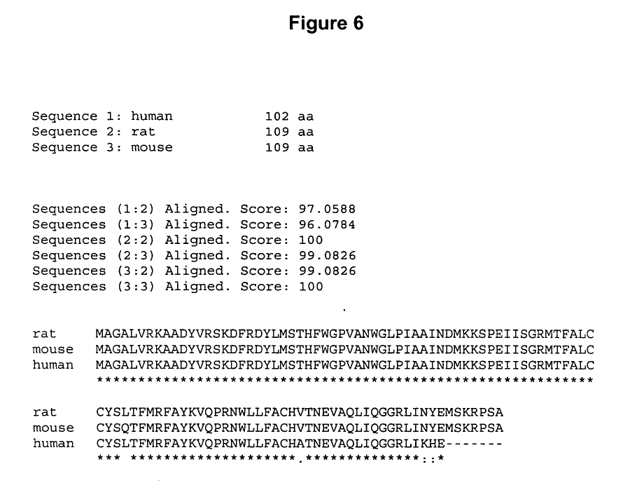

The amino acid sequence of the polypeptide SEQ ID No. 4 is shown in FIG. 5. The predicted amino acid sequence has 91% identity and 95% homology with the predicted amino acid sequence of the human polypeptide encoded by the nucleic acid sequence of GenBank Accession number NM_{—}016098, and 99% identity and 99% homology with the predicted amino acid sequence of the mouse polypeptide encoded by the nucleic acid sequence of GenBank Accession number NM_{—}018819 as shown in FIG. 6.

A polypeptide including an amino acid sequence of SEQ ID No. 4 in the various forms of the present invention includes within its scope the 109 amino acid polypeptide itself, the fusion of any other amino acid sequences to the amino and/or carboxy terminal of the polypeptide, and any other variant substantially identical to SEQ ID No. 4, such as those variants that have one or more substituted amino acids that still retain the same biological activity of regulating apoptosis and/or cell proliferation.

A polypeptide including an amino acid sequence at least 90% homologous to an amino acid sequence of SEQ ID No. 4 in the various forms of the present invention includes within its scope any homologue or other similar protein with at least 90% sequence homology to SEQ ID No. 4. Examples of such polypeptides include the predicted human polypeptide encoded by the nucleic acid of GenBank Accession number NM_{—}016098 (95% homology with SEQ ID No. 4), or the predicted mouse polypeptide encoded by the nucleic acid of GenBank Accession number NM_{—}018819 (99% homology with SEQ ID No. 4).

Preferably, the polypeptide has an amino acid sequence that is at least 95% homologous to the amino acid sequence of SEQ ID No. 4.

More preferably, the polypeptide has an amino acid sequence that is at least 90% identical to the amino acid sequence of SEQ ID No. 3. Most preferably, the polypeptide has an amino acid sequence that is at least 95% identical to the amino acid sequence of SEQ ID No. 4.

Various algorithms exist for determining the degree of homology between any two proteins. For example, the BLAST and FASTA algorithms can be used for determining the extent of amino acid sequence homology between two sequences. BLAST identifies local alignments between the sequences in the database and predicts the probability of the local alignment occurring by chance. The BLAST algorithm is as described in Altschul et al., 1990, J. Mol. Biol. 215:403-410.

A polypeptide including an amino acid sequence of a biologically active fragment of a polypeptide having an amino acid sequence of SEQ ID No. 4 (or a polypeptide having an amino acid sequence 90% homologous to SEQ ID NO:4) in the various forms of the present invention includes any fragment of the SEQ ID No. 4 polypeptide or a homologue that produces a functional protein able to regulate apoptosis and/or cell proliferation. As will be appreciated, this will include truncations and deletions of the polypeptide or homologue that still retain biological activity, and chimeric polypeptides containing a biologically active fragment of the SEQ ID No. 4 polypeptide or homologue fused to one or more amino acids.

An alteration of the expression or function in the cell of a polypeptide may be accomplished in a number of different ways known in the art. For example, increasing the expression of a polypeptide in the cell may be achieved by transforming the cell with a recombinant nucleic acid encoding the polypeptide of interest under the control of a promoter that is active in the particular cell type. In this case, a suitable vector for expressing a nucleic acid encoding a sequence based on SEQ ID NO.4 in a large variety of cell types is the p3.1 cDNA vector. In this case, expression is driven from the CMV promoter.

A decrease in expression of the level of a polypeptide in a cell may be accomplished by the use of an antisense nucleic acid that binds to an endogenous mRNA and which interferes with translation, the use of a molecule that can specifically repress transcription of an endogenous mRNA such as a specific DNA or RNA binding protein, a nucleic acid capable of forming a triple helix structure, a small interfering RNA, or a ribozyme that can cleave a specific mRNA.

In this regard, the process of transformation will be understood to be the process by which exogenous DNA enters a recipient eukaryotic cell. It may occur under natural or artificial conditions using various methods known in the art, including transformation using calcium phosphate, viral infection, electroporation, lipofection, and particle bombardment. Transformed cells include stably transformed cells in which the inserted DNA is capable of replication either as an autonomously replicating plasmid or as part of the host chromosome, or cells which transiently express the inserted DNA or RNA for limited periods of time. Methods for introducing exogenous DNAs into cells are essentially as described in Sambrook, J, Fritsch, E. F. and Maniatis, T. Molecular Cloning: A Laboratory Manual 2nd. ed. Cold Spring Harbor Laboratory Press, New York. (1989).

An alteration in the function of a polypeptide may be accomplished in a number of different ways known in the art. For example, a molecule that binds to, or interacts with, the polypeptide to either inhibit or promote its activity may be used. For example, a molecule capable of binding to the polypeptide may be contacted with the cell, be taken up by the cell and bind to the polypeptide to alter its ability to function. Types of molecules capable of such actions include proteins, small molecules, drugs, substrate analogues, transition state analogues, antibodies including neutralizing antibodies, and nucleic acids such as aptamers. Alternatively, a molecule may be expressed in the cell that has the capacity to bind or interact with the polypeptide.

The present invention also provides a method of regulating apoptosis in a cell, the method including the step of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80% identical to a nucleotide sequence complementary to SEQ ID No 3, or RNA equivalent thereof;

(e) a nucleotide sequence encoding a biologically active fragment of a polypeptide having an amino acid sequence of SEQ ID No.4;

(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

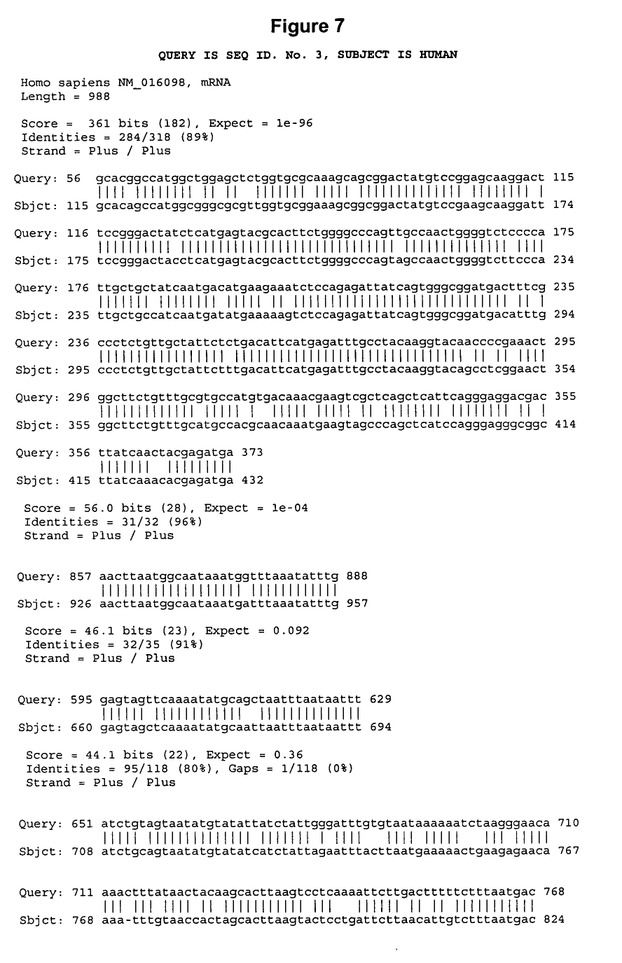

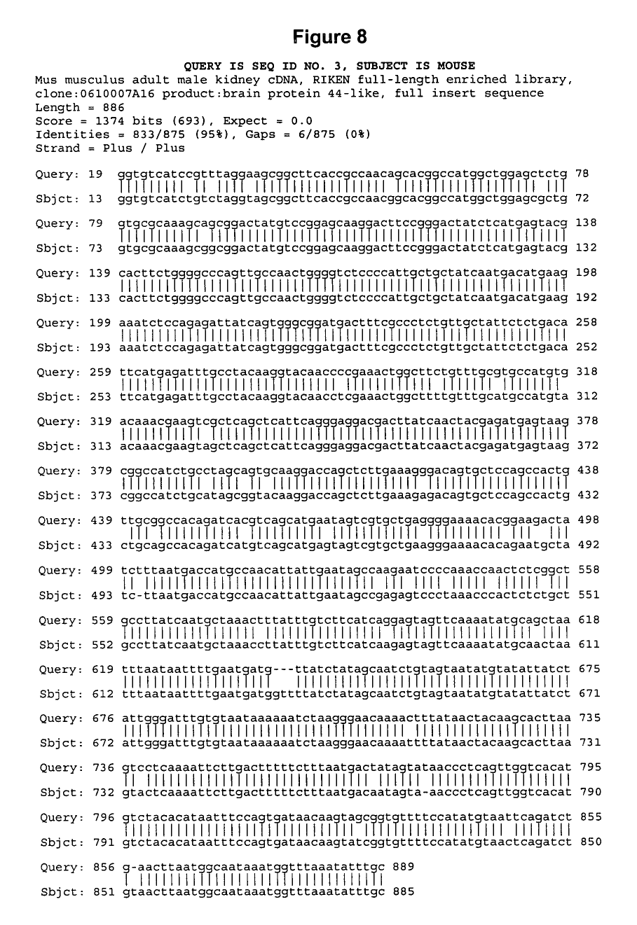

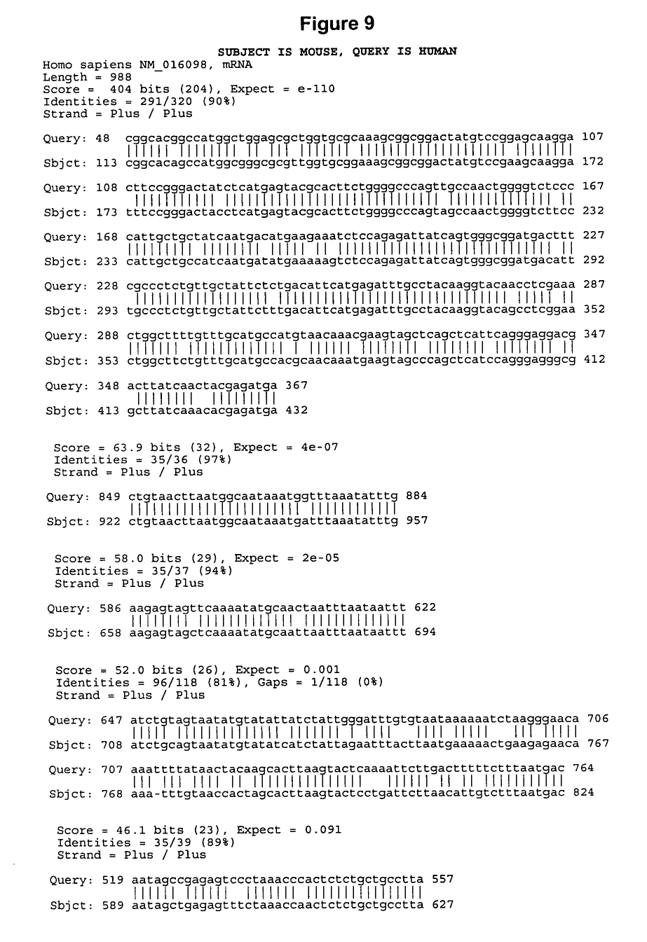

The nucleotide sequence of SEQ ID No. 3 is shown in FIG. 5. The nucleotide sequence has 89% (average) identity with the nucleotide sequence of Genbank accession No. NM_{—}016098 encoding the human homologue as shown in FIG. 7. The nucleotide sequence of SEQ ID No. 3 also has 95% identity with the nucleotide sequence of GenBank accession No. NM_{—}018819 of the mouse homologue as shown in FIG. 8. The nucleotide sequence of the human (NM-016098) is 90% identical with the nucleotide sequence of the mouse (NM-018819), as shown in FIG. 9.

The method of this form of the present invention provides the ability to regulate apoptosis by the expression in a cell of various nucleic acids based on SEQ ID No. 3 (or a nucleotide sequence based on that encoding the amino acid sequence of SEQ ID No. 4). This form of the present invention also contemplates cells transformed with such nucleic acids.

For example, apoptosis of cells may be inhibited by a nucleic acid that results in a reduction in ARBP expression in cells.

The cell is any eukaryotic cell that has the capacity to undergo apoptosis. For example, the cell may be a human cell, a mouse cell or a rat cell. An example of a cell type for which apoptosis may be regulated by the method of the present invention is a neuronal cell.

The cell may be a cell present in an entire organism, such as an animal or human, or alternatively may be a cell cultured in vitro, such as a cell grown in tissue culture. For example, the cell may be an isolated cell (such as a human or rat neuronal cell), a cell that has the capacity to undergo apoptosis present in a tissue or organ in an animal or human subject, or a cell that is undergoing apoptosis present in a tissue or organ in an animal or human subject. The cell may also be a cell having dysregulation of apoptosis or a cell associated with a disease or condition involving dysregulation of apoptosis.

Examples of cells for which apoptosis may be regulated include cells in the ventral horn (neurons), adrenal medulla (neurons), adrenal cortex, dorsal root ganglion, trigeminal ganglia, hypoglossal nucleus, peripheral nerve (Schwann cells), caudal ventral lateral medulla, cerebellar deep nuclei, singular cortex, dorsal column, dorsal horn, footpad sweat gland, gut, hippocampus, kidney medulla, liver, lung, lymph nodes, mid brain, salivary gland, skin, spleen, substantia nigra, sweat gland, testes, and thymus.

Preferably the cell is a neuronal cell. More preferably, the neuronal cell is a rat or human neuronal cell. Examples of neuronal cells include neuronal cells present in substantia nigra, dorsal root ganglia, trigeminal ganglia and olfactory bulb, motor neurons and hypoglossal neurons.

Examples of non-neuronal cell types for which apoptosis may be regulated include non-neuronal cells present in neural and brain tissue, intestinal cells including goblet cells, lymph node cells, spleen cells, liver cells, thymic cells, salivary gland cells, pituitary cells, bladder cells, bone cells, breast cells, cervical cells, colorectal cells, kidney cells, laryngeal cells, blood cells, lung cells, lymphatic cells, skin cells, plasma cells, muscle cells, cells of the mouth or throat, ovaries, pancreatic cells, prostate cells, stomach cells, testicular cells, germ cells, thyroid cells, and uterine cells.

Preferably the cell has the capacity to undergo apoptosis mediated by a neurotrophin receptor and/or the binding of a neurotrophin to a receptor. Examples of neurotrophin receptors include TrkA, TrkB, TrkC, and p75NTR. More preferably, the cell has the capacity to undergo apoptosis mediated by p75NTR, or a cell that has the capacity to undergo apoptosis mediated by the binding of a neurotrophin to p75NTR. Examples of neurotrophins that have the capacity to bind to p75NTR include NGF, BDNF, NT-3, NT-4/NT-5 and NT-6.

Preferably, the cell is a neuronal cell that has the capacity to undergo apoptosis mediated by a neurotrophin receptor and/or the binding of a neurotrophin to a receptor.

Accordingly, in a preferred embodiment, the present invention provides a method of regulating apoptosis of a neuronal cell mediated by a neurotrophin receptor and/or a neurotrophin binding to a receptor, the method including the steps of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80% identical to a nucleotide sequence complementary to SEQ ID No 3, or RNA equivalent thereof;

(e) a nucleotide sequence encoding a biologically active fragment of a polypeptide having an amino acid sequence of SEQ ID No. 4;

(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

The neurotrophin receptor may be a receptor normally expressed endogenously in the cell of interest, or may be a receptor introduced into a cell, for example by way of transient or stable transformation, or by viral infection or transduction, by methods known in the art.

The neurotrophin receptor introduced into the cell may be from the same species or from a different species as the recipient cell, and may consist of the entire receptor or a biologically active fragment or variant of the receptor. For example, the entire human neurotrophin receptor p75NTR may be introduced into a rat neuronal cell.

The neurotrophin may be any neurotrophin that may bind to a receptor present in a cell and cause apoptosis. Preferably, the neurotrophin is NGF, BDNF, NT-3, NT-4/NT-5 and NT-6.

The expression in the cell of the nucleic acid may be by a suitable method known in the art. For example, a nucleic acid including a sequence based on SEQ ID No. 3 may be isolated and cloned into a suitable expression vector for use in the cell type of interest by methods known in the art. Methods for the isolation of nucleic acid sequences based on SEQ ID No. 3 and their cloning into a suitable expression vector are essentially as described in Sambrook, J, Fritsch, E. F. and Maniatis, T. Molecular Cloning: A Laboratory Manual 2nd. ed. Cold Spring Harbor Laboratroy Press, New York. (1989). The recombinant molecule may then be introduced into the cell and the cloned nucleic acid expressed.

As will be appreciated, the level of expression of the nucleic acid in the cell will be at a sufficient level to achieve a desired level of regulation of apoptosis in the particular cell type. Accordingly, expression of the nucleic acid will be from a suitable constitutive or inducible promoter known in the art.

For example, a suitable vector for expressing a nucleic acid including a sequence based on SEQ ID NO.3 in a large variety of cell types is the p3.1cDNA vector. In this case, expression is driven from the CMV promoter.

A nucleic acid including a nucleotide sequence of SEQ ID No. 3 in the various forms of the present invention includes within its scope all DNA and RNA forms of the nucleotide sequence. As will be understood, by virtue of the degeneracy of the genetic code, SEQ ID No. 3 will also include within its scope similar nucleic acid sequences encoding the same protein. Thus, the invention contemplates for example variations of nucleotide sequence that could be made by selecting combinations based on possible codon choices. These combinations are made in accordance with the standard triplet genetic code as applied to the nucleotide sequence of the coding region of SEQ ID No. 3.

A nucleic acid including a nucleotide sequence at least 80% identical to a nucleotide sequence of SEQ ID No. 3 in the various forms of the present invention includes within its scope all DNA and RNA forms that are at least 80% identical to the SEQ ID No. 3 nucleotide sequence. Thus, the nucleic acid includes within its scope natural and recombinant DNA homologues of SEQ ID No. 3, and any other variants of the primary sequence of SEQ ID No. 3. Examples of such nucleic acids include the human nucleic acid of GenBank Accession number NM_{—}016098 (89% with SEQ ID No. 3), or the mouse nucleic acid of GenBank Accession number NM_{—}018819 (95% homology with SEQ ID No. 3).

Preferably the nucleotide sequence has at least 90% identity to the nucleotide sequence of SEQ ID No. 3. More preferably, the nucleotide sequence has at least 95% identity to the nucleotide sequence of SEQ ID No. 3.

In this regard, various algorithms exist for determining the degree of homology between any two nucleic acid sequences. For example, the BLAST algorithm can be used for determining the extent of sequence homology between two sequences. BLAST identifies local alignments between two sequences and predicts the probability of the local alignment occurring by chance. The BLAST algorithm is as described in Altschul et al., 1990, J. Mol. Biol. 215:403-410.

A nucleic acid including a nucleotide sequence complementary to SEQ ID No. 3 in the various forms of the present invention includes within its scope all DNA and RNA forms of the complementary nucleotide sequence. The expression of such a nucleic acid in a cell is useful, for example, for the expression of antisense nucleic acids that interfere with the expression of the endogenous ARBP protein or homologues of the protein.

A nucleic acid including a nucleotide sequence at least 80% identical to a nucleotide sequence complementary to SEQ ID No. 3 in the various forms of the present invention includes within its scope all DNA and RNA forms that are at least 80% identical to the complementary nucleotide sequence. The expression of such a nucleic acid in a cell is also useful for the expression of antisense nucleic acids that interfere with the expression of endogenous ARBP protein or homologues of the protein.

Preferably, the nucleotide sequence has at least 90% identity to a nucleotide sequence complementary to SEQ ID No. 3. More preferably, the nucleotide sequence has at least 95% identity to a nucleotide sequence complementary to SEQ ID No. 3.

A nucleic acid sequence encoding a biologically active fragment of a polypeptide having an amino acid sequence of SEQ ID No. 4 in the various forms of the present invention includes within its scope any nucleic acid which encodes a polypeptide that is a fragment of SEQ ID No. 4, such fragment retaining the biological activity of the full length protein (ie the ability to regulate apoptosis and/or cell proliferation), or a variant thereof. For example, such nucleic acids may be nucleic acid sequences encoding amino and/or carboxy terminal deletions of the full length protein, nucleic acid sequences encoding internal deletions of the full length protein, or nucleic acid sequences encoding chimeric polypeptides containing a biologically active fragment of the SEQ ID No. 4 polypeptide fused to one more amino acids.

A nucleic acid including a nucleotide sequence encoding an antisense nucleic acid that reduces the expression in the cell of a polypeptide including an amino acid sequence of SEQ ID No. 4, or an amino acid sequence at least 90% identical to an amino acid sequence of SEQ ID No. 4, in the various forms of the present invention is any nucleic acid that acts as an antisense nucleic acid to reduce the expression in the cell of a polypeptide including the amino acid sequence of SEQ ID No. 4 or an amino acid sequence at least 90% identical to an amino acid sequence of SEQ ID No. 4.

Such a nucleic acid can be used to inhibit apoptosis by decreasing the expression in a cell of polypeptides based on ARBP.

Accordingly, in a preferred form, the present invention provides an antisense nucleic acid that reduces the expression in a cell of a polypeptide including an amino acid sequence of SEQ ID NO. 4 or an amino acid sequence at least 90% identical to an amino acid sequence of SEQ ID NO. 4.

As will be appreciated, the antisense nucleic acid will be expressed in the cell at a sufficient level to reduce the expression of the SEQ ID No. 4 polypeptide or a polypeptide including an amino acid sequence at least 90% identical to SEQ ID No. 4. As described previously, an example of a vector suitable for the purpose of expressing an antisense nucleic acid is p3.1cDNA.

The antisense nucleic acid will include a sequence complementary to at least a portion of the target RNA encoding SEQ ID No. 4 or a homologue thereof. Absolute complementarity, although preferred, is not required, as long as the antisense nucleic acid is capable of hybridizing with the target RNA and thereby interferes with expression from the RNA. As will be appreciated, the ability to hybridize will depend on both the degree of complementarity and the length of the antisense nucleic acid. Methods known in the art may be used to formulate possible antisense nucleic acids.

Preferably, the nucleotide sequence encoding an antisense nucleic acid includes a sequence having at least 15 contiguous nucleotides identical to SEQ ID No. 3. More preferably, the nucleotide sequence encoding an antisense nucleic acid includes a sequence having at least 50 contiguous nucleotides identical to SEQ ID No. 3. More preferably, the nucleotide sequence encoding an antisense nucleic acid includes a sequence having at least 500 contiguous nucleotides identical to SEQ ID No. 3.

Methods for determining the ability of a nucleotide sequence to act as an antisense nucleic acid may be by a suitable method known in the art. For example, the steady state levels of a target polypeptide in the presence and absence of the antisense nucleic acid may be determined. In this case, the level of polypeptide may be determined in a cell stably transformed with a vector expressing the antisense nucleic acid and in a cell stably transformed with a vector that does not express the antisense nucleic acid. Detection and measurement of the protein concentration (eg by Western analysis with an appropriate antibody) may reveal that the level of protein expressed in the cell is reduced in the cell in which the antisense nucleic acid is present.

In vitro methods known in the art may also be used to confirm that a nucleic acid is able to function as an antisense nucleic acid. For example, a cell free extract capable of in vitro translation may be prepared, and the ability of the nucleic acid to act as an antisense can be confirmed by a reduction in the level of protein when the extract is used to direct translation. The capacity of the nucleic acid to hybridise to the appropriate mRNA for which translation is being inhibited can be confirmed by the use of suitable Rnases that specifically recognise and cleave the double stranded complex.

As will be appreciated, the antisense nucleic acid may also be an antisense oligonucleotide capable of reducing the expression in the cell of a polypeptide including an amino acid sequence of SEQ ID No. 4 or an amino acid sequence at least 90% identical to an amino acid sequence of SEQ ID No. 4. Such oligonucleotides may be produced by a method known in the art, including chemical synthesis. The oligonucleotide may then be introduced into a cell by a method known in the art.

A nucleotide sequence encoding a ribozyme that cleaves a mRNA encoding an amino acid sequence of SEQ ID No. 4, or an amino acid sequence 90% identical to an amino acid sequence of SEQ ID No. 4, in the various forms of the present invention includes within its scope any nucleic acid sequence that acts as a ribozyme that specifically cleaves such a mRNA.

The functional constraints necessary for a nucleic acid to act as a ribozyme are essentially as described in Haseloff et al., (1988) Nature 334: 585-591; Koizumi et al., (1988) FEBS Lett., 228: 228-230; Koizumi et al., (1988)FEBS Lett., 239: 285-288). Ribozyme methods that involve inducing expression in a cell ribozyme molecules are essentially as described in Grassi and Marini (1996) Annals of Medicine 28: 499-510 ; Gibson (1996) Cancer and Metastasis Reviews 15: 287-299.

The mechanism of ribozyme action involves sequence-specific hybridization of the ribozyme molecule to complementary target RNA, followed by endonucleolytic cleavage. Examples of ribozymes which may be used include engineered hammerhead motif ribozyme molecules that can specifically and efficiently catalyze endonucleolytic cleavage of sequences encoding the target sequence.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays, as known in the art.

The ribozyme according to this form of the present will be expressed in vivo or introduced into a cell as a synthetic ribozyme, in sufficient number to be catalytically effective in cleaving mRNA, and thereby modify mRNA abundance in a cell. For example, a ribozyme coding DNA sequence may be synthesized and ligated into a restriction enzyme site in the anticodon stem and loop of a gene encoding a tRNA, which can then be transformed into and expressed in a cell of interest by a method known in the art. Alternately, an inducible promoter can by used so that ribozyme expression can be selectively controlled in a particular cell type.

Methods for determining the ability of a nucleic acid to function as a ribozyme include assays known in the art to determine the ability of a nucleic acid to function as a ribozyme in vitro, or methods to determine the level of inhibition of gene expression in vivo by the ribozyme.

In another embodiment, the present invention also provides a synthetic ribozyme that cleaves a mRNA encoding an amino acid sequence of SEQ ID No. 4 or an amino acid sequence at least 90% identical to an amino acid sequence of SEQ ID No. 4.

Such ribozymes may produced by a method known in the art, including in vitro chemical synthesis. The ribozyme may be introduced into the cell by a method known in the art.

The present invention further provides a vector including a nucleic acid sequence selected from the group consisting of:

(a) a nucleic acid having a nucleotide sequence of SEQ ID No. 3;

(b) a nucleic acid having a nucleotide sequence at least 80% identical to a nucleotide sequence of SEQ ID No. 3;

(c) a nucleic acid having a nucleotide sequence complementary to SEQ ID No. 3;

(d) a nucleic acid having a nucleotide sequence at least 80% identical to a nucleotide sequence complementary to SEQ ID No. 3;

(e) a nucleic acid sequence encoding a biologically active fragment of a polypeptide having an amino acid sequence of SEQ ID No. 4;

(f) a nucleic acid encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleic acid encoding a ribozyme that cleaves a mRNA encoding an amino acid of SEQ ID No. 4 or an amino acid sequence 90% identical to an amino acid sequence of SEQ ID No. 4.

The vector may be any nucleic acid capable of having a foreign nucleic acid inserted into the vector. For example, the vector may be a plasmid, all or part of a viral genome, or any other nucleic acid capable of autonomous replication in a prokaryotic or eukaryotic host.

The vector may also further include regulatory elements for the expression of inserted nucleic acids, for example promoters for driving the expression of an inserted nucleic acid in a particular cell, poly A signals for efficient polyadenylation of mRNA transcribed from inserted nucleic acids, or other regulatory elements to control translation, transcription or mRNA stability.

Preferably, the vector includes a nucleic acid sequence encoding a promoter functional in an animal or human cell. More preferably, the vector is for the expression of the cloned nucleic acid in brain cells and therefore includes a nucleic acid sequence encoding a promoter that is functional in brain cells. In this case, preferably the vector includes a nucleic acid sequence encoding a promoter functional in a neuronal cell.

In another embodiment, the present invention also provides a prokaryotic or eukaryotic cell transformed with such a vector. Preferably, the cell is an animal or human cell. The cell may be a neuronal or non-neuronal cell. Most preferably, the cell is a neuronal cell.

The present invention further provides a method of regulating proliferation of a cell, the method including the step of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% identical to an amino acid sequence of SEQ ID No. 4, or biologically active fragment thereof.

The method of this form of the present invention provides the ability to regulate the proliferation of a cell by altering the expression and/or function of a polypeptide including an amino acid sequence selected from the group consisting of: (i) an amino acid sequence of SEQ ID No. 4; (ii) an amino acid sequence of a biologically active fragment of a polypeptide having an amino acid sequence of SEQ ID No. 4.; and (iii) an amino sequence at least 90% identical to an amino acid sequence of SEQ ID No. 4; or a biologically active fragment thereof.

In this regard, it has been found that altering the expression of the ARBP protein by antisense RNA expression can regulate cell proliferation. In particular, it has been found that cell proliferation in a transgenic neural cell line expressing a human neurotrophin receptor may be regulated by altering the expression of ARBP.

The cell is any eukaryotic cell that has the capacity to undergo proliferation, or a cell that is actively proliferating. For example, the cell may be a human cell, a mouse cell or a rat cell. An example of a cell type for which proliferation may be regulated by the method of the present invention is a neuronal cell.

The cell may be present in an entire organism, such as an animal or human, or alternatively may be a cell cultured in vitro, such as a cell grown in tissue culture. For example, the cell may be an isolated cell (such as an isolated rat neuronal cell), a cell that has the capacity to proliferate that is present in a tissue or organ in an animal or human subject, or a cell that is undergoing proliferation that is present in a tissue or organ in an animal or human subject.

Examples of cells for which proliferation may be regulated include cells in the ventral horn (neurons), adrenal medulla (neurons), adrenal cortex, dorsal root ganglion, trigeminal ganglia, hypoglossal nucleus, peripheral nerve (Schwann cells), caudal ventral lateral medulla, cerebellar deep nuclei, singular cortex, dorsal column, dorsal horn, footpad sweat gland, gut, hippocampus, kidney medulla, liver, lung, lymph nodes, mid brain, salivary gland, skin, spleen, substantia nigra, sweat gland, testes, and thymus.

For example, the cell may be a neuronal cell, a cell present in neural tissue, an intestinal cell, a lymph node cell, a spleen cell, a liber cell, a thymic cell, and a salivary gland cell. Cells present in neural tissues include Schwann cells, motor neuron cells, substantia nigra cells, and pituitary cells.

Preferably the cell is a neuronal cell. More preferably, the neuronal cell is a rat or human neuronal cell. Examples of neuronal cells include neuronal cells present in substantia nigra, dorsal root ganglia, trigeminal ganglia and olfactory bulb, motor neurons and hypoglossal neurons.

Examples of non-neuronal cell types for which proliferation may be regulated include non-neuronal cells present in neural and brain tissue, intestinal cells including goblet cells, lymph node cells, spleen cells, liver cells, thymic cells, salivary gland cells, pituitary cells, bladder cells, bone cells, breast cells, cervical cells, colorectal cells, kidney cells, laryngeal cells, blood cells, lung cells, lymphatic cells, skin cells, plasma cells, muscle cells, cells of the mouth or throat, ovaries, pancreatic cells, prostate cells, stomach cells, testicular cells, germ cells, thyroid cells, and uterine cells.

Preferably, the cell has the capacity to undergo proliferation mediated by a neurotrophin receptor and/or the binding of a neurotrophin to a receptor. Examples of neurotrophin receptors include TrkA, TrkB, TrkC, and p75NTR. More preferably, the cell has the capacity to undergo proliferation mediated by p75NTR, or a cell that has the capacity to undergo apoptosis mediated by the binding of a neurotrophin to p75NTR. Examples of neurotrophins that have the capacity to p75NTR include NGF, BDNF, NT-3, NT-4/NT-5 and NT-6.

Preferably, the cell is a neuronal cell that has the capacity to undergo proliferation mediated by a neurotrophin receptor and/or the binding of a neurotrophin to a receptor.

Accordingly, in a preferred embodiment, the present invention provides a method of regulating proliferation of a neuronal cell mediated by a neurotrophin receptor and/or binding of a neurotrophin to a receptors, the method including the steps of altering the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% homologous to an amino acid sequence of SEQ ID No. 4, or a biologically active fragment thereof.

The neurotrophin receptor may be a receptor normally expressed endogenously in the cell of interest, or may be a receptor introduced into a cell, for example by way of transient or stable transformation, or by viral infection or transduction, by methods known in the art.

A neurotrophin receptor introduced into the cell may be from the same species or from a different species as the recipient cell, and may consist of the entire receptor or a biologically active fragment or variant of the receptor. For example, the entire human neurotrophin receptor p75NTR may be introduced into a rat neuronal cell.

The neurotrophin may be any neurotrophin that may bind to a receptor present in a cell and cause apoptosis. Preferably, the neurotrophin is NGF, BDNF, NT-3, NT-4/NT-5 and NT-6.

The regulation of cell proliferation in the various forms of the present invention is any form of control or change in the proliferation of a cell. For example, regulation of proliferation may involve (i) inhibiting or inducing the ability of a cell to begin proliferating; (ii) inhibiting or inducing the proliferation in a cell after proliferation has actively begun apoptosis; and/or (iii) reduce or promote the probability that a particular cell will begin or continue proliferating.

The cell proliferation in the various forms of the present invention may be any form of induction or inhibition of proliferation or that occurs in a cell in response to any genetic or external response.

In one form, the present invention provides a method of promoting proliferation of a cell, the method including the step of decreasing the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino acid sequence at least 90% identical to an amino acid sequence of SEQ ID No. 4, or biologically active fragment thereof.

Methods for determining the extent of cell proliferation in the various forms of the present invention may be performed by a suitable method known in the art, including (i) monitoring cell number by trypan blue staining; (ii) assays measuring the number of actively dividing cells, for example by the measurement of DNA synthesis by 3[H] thymidine uptake; and (iii) the measurement of metabolic activity, for example as determined by the use of tetrazolium salts, essentially as described in Marks D. C. et al. (1992) Leuk. Res 16:1165-1173.

The present invention also provides a method of regulating the proliferation of a cell, the method including the step of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80%, identical to a nucleotide sequence complementary to SEQ ID No. 3, or RNA equivalent thereof;

(e) a nucleotide sequence encoding a biologically active fragment of a polypeptide having an amino acid sequence of SEQ ID No. 4;

(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

The method of this form of the present invention provides the ability to regulate proliferation of a cell by the expression of various nucleic acids based on SEQ ID No. 3 (or a nucleotide sequence based on that encoding the amino acid sequence of SEQ ID No. 4).

For example, the proliferation of cells may be promoted by a nucleic acid that results in a reduction in ARBP expression in cells.

The cell is any eukaryotic cell that has the capacity to undergo proliferation or a cell that is actively proliferating. For example, the cell may be a human cell, a mouse cell or a rat cell. An example of a cell type for which proliferation may be regulated by the method of the present invention is a neuronal cell.

The cell may be present in an entire organism, such as an animal or human, or alternatively may be a cell cultured in vitro, such as a cell grown in tissue culture. For example, the cell may be an isolated cell (such as a human or rat neuronal cell), a cell that has the capacity to proliferate present in a tissue or organ in an animal or human subject, or a cell that is undergoing proliferation present in a tissue or organ in an animal or human subject.

Examples of cells for which proliferation may be regulated include cells in the ventral horn (neurons), adrenal medulla (neurons), adrenal cortex, dorsal root ganglion, trigeminal ganglia, hypoglossal nucleus, peripheral nerve (Schwann cells), caudal ventral lateral medulla, cerebellar deep nuclei, singular cortex, dorsal column, dorsal horn, footpad sweat gland, gut, hippocampus, kidney medulla, liver, lung, lymph nodes, mid brain, salivary gland, skin, spleen, substantia nigra, sweat gland, testes, and thymus.

Preferably the cell is a neuronal cell. More preferably, the neuronal cell is a rat or human neuronal cell. Examples of neuronal cells include neuronal cells present in substantia nigra, dorsal root ganglia, trigeminal ganglia and olfactory bulb, motor neurons and hypoglossal neurons.

Examples of non-neuronal cell types for which proliferation may be regulated include non-neuronal cells present in neural and brain tissue, intestinal cells including goblet cells, lymph node cells, spleen cells, liver cells, thymic cells, salivary gland cells, pituitary cells, bladder cells, bone cells, breast cells, cervical cells, colorectal cells, kidney cells, laryngeal cells, blood cells, lung cells, lymphatic cells, skin cells, plasma cells, muscle cells, cells of the mouth or throat, ovaries, pancreatic cells, prostate cells, stomach cells, testicular cells, germ cells, thyroid cells, and uterine cells.

Preferably, the cell is a cell that has the capacity to undergo proliferation mediated by a neurotrophin receptor or the binding of a neurotrophin to a receptor. Examples of neurotrophin receptors include TrkA, TrkB, TrkC, and p75NTR. More preferably, the cell has the capacity to undergo proliferation mediated by p75NTR, or a cell that has the capacity to undergo proliferation mediated by the binding of a neurotrophin to p75NTR. Examples of neurotrophins that have the capacity to p75NTR include NGF, BDNF, NT-3, NT-4/NT-5 and NT-6.

Preferably the cell is a neuronal cell that has the capacity to undergo proliferation mediated by a neurotrophin receptor and/or the binding of a neurotrophin to a receptor.

Accordingly, in a preferred form, the present invention provides a method of regulating proliferation of a neuronal cell mediated by a neurotrophin receptor and/or binding of a neurotrophin to a receptor, the method including the steps of expressing in the cell a nucleic acid including a nucleotide sequence selected from the group consisting of:

(a) a nucleotide sequence of SEQ ID No. 3, or RNA equivalent thereof;

(b) a nucleotide sequence at least 80% identical to a nucleotide sequence of SEQ ID No. 3, or RNA equivalent thereof;

(c) a nucleotide sequence complementary to SEQ ID No. 3, or RNA equivalent thereof;

(d) a nucleotide sequence at least 80% identical to a nucleotide sequence complementary to SEQ ID No. 3, or RNA equivalent thereof;

(e) a nucleotide sequence encoding a biologically active fragment of a polypeptide having an amino acid sequence of SEQ ID No. 4;

(f) a nucleotide sequence encoding an antisense nucleic acid that reduced the expression in the cell of a polypeptide including an n amino acid sequence of SEQ ID No. 4 or an amino acid sequence at least 90% identical to an amino acid sequence of SEQ ID No. 4; and

(g) a nucleotide sequence encoding a ribozyme that cleaves a mRNA encoding an amino acid of SEQ ID No. 4 or an amino acid sequence 90% identical to an amino acid sequence of SEQ ID No. 4.

The neurotrophin receptor may be a receptor normally expressed endogenously in the cell of interest, or may be a receptor introduced into a cell, for example by way of transient or stable transformation, or by viral infection or transduction, by methods known in the art.

A neurotrophin receptor introduced into the cell may be from the same species or from a different species, and may consist of the entire receptor or a biologically active fragment or variant of the receptor. For example, the entire human neurotrophin receptor p75NTR may be introduced into a rat neuronal cell.

The neurotrophin may be any neurotrophin that may bind to a receptor and cause proliferation. Examples of neurotrophins include NGF, BDNF, NT-3, NT-4/NT-5 and NT-6.

The expression in the cell of the nucleic acid may be by a suitable method known in the art. For example, the nucleic acid including a sequence based on SEQ ID No. 3 may be isolated and cloned into a suitable expression vector for use in the cell type of interest by methods known in the art. Methods for the isolation of nucleic acid sequences based on SEQ ID No. 3 and their cloning into a suitable expression vector are as described in Sambrook, J, Fritsch, E. F. and Maniatis, T. Molecular Cloning: A Laboratory Manual 2nd. ed. Cold Spring Harbor Laboratory Press, New York. (1989). The recombinant molecule may then be introduced into the cell and the cloned nucleic acid expressed.

As will be appreciated, the level of expression of the nucleic acid in the cell will be at a sufficient level to achieve the desired level of regulation of proliferation in the particular cell type. Accordingly, expression of the nucleic acid will be from a suitable constitutive or inducible promoter known in the art.

For example, a suitable vector for expressing a nucleic acid including a sequence based on SEQ ID NO. 3 in a large variety of cell types is the p3.1 cDNA vector. In this case, expression is driven from the CMV promoter.

The present invention also provides an agent for regulating cell apoptosis and/or proliferation, wherein the administration of an effective amount of the agent to a cell alters the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino sequence at least 90% identical to an amino acid sequence of SEQ ID No. 4, or a biologically active fragment thereof.

The agent that regulates cell apoptosis and/or proliferation according to the various forms of the present invention is any agent that when administered to the cell alters the expression and/or function in the cell of a polypeptide based on the amino acid sequence of SEQ ID No. 4.

In this regard, the term “administration” will be understood to include within its scope the external administration of the agent to a cell or the intracellular expression of the agent in the cell. Accordingly, the administration may be by way of contacting the cell with the agent, or for example, by way of transforming the cell with a recombinant nucleic acid capable of directing the expression of the agent in the cell.

In the case of extra-cellular administration of the agent to the cell, the agent may be a nucleic acid, an oligonucleotide, a polypeptide, a peptide, an enzyme, a small molecule, a drug, a polysaccharide, a glycoprotein, a hormone, a receptor, a ligand for a receptor, a small interfering RNA, or a lipid. In the case of intra-cellular administration, the agent may be a nucleic acid, a polypeptide, an enzyme, a small molecule, a polysaccharide, a hormone, a receptor, an antibody including a neutralising antibody, a ligand for a receptor, or a lipid.

Preferably, the agent is a nucleic acid. More preferably, the agent is an antisense nucleic acid, a ribozyme, a small interfering RNA or an aptamer. Most preferably, the agent is an antisense nucleic acid. In this regard, it has been found that the expression in a cell of a nucleic acid including a nucleotide sequence complementary to SEQ ID No. 3 is capable of acting as an antisense molecule to reduce expression and thereby regulate apoptosis or cell proliferation.

Accordingly, in a preferred form the present invention provides an agent for inhibiting cell apoptosis and/or promoting cell proliferation, wherein the administration of an effective amount of the agent to a cell decreases the expression and/or function in the cell of a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino sequence at least 90% identical to an amino acid sequence of SEQ ID No. 4, or a biologically active fragment thereof.

In another form, the present invention provides an antisense nucleic acid that reduces the expression in a cell of a polypeptide including an amino acid sequence of SEQ ID No. 4 or an amino acid sequence at least 90% identical to an amino acid sequence of SEQ ID No. 4.

In the case where the agent is an antisense nucleic acid, the nucleic acid may be a nucleic acid complementary to all or part of the nucleotide sequence of SEQ ID No.3. For example, the antisense nucleic acid may be a nucleic acid expressed intracellularly, such as a nucleotide sequence complementary to SEQ ID No. 3, or an exogenous oligonucleotide complementary to part of SEQ ID No. 3.

Preferably, the antisense nucleic acid includes a sequence having at least 15 contiguous nucleotides identical to SEQ ID No. 3. More preferably, the antisense nucleic acid includes a sequence having at least 50 contiguous nucleotides identical to SEQ ID No. 3. Most preferably, the antisense nucleic acid includes a sequence having at least 500 contiguous nucleotides identical to SEQ ID No. 3.

The antisense nucleic acid may be composed of DNA or RNA, or any modification or derivative thereof. The antisense nucleic acid may also be an oligonucleotide or a polynucleotide. In a preferred form of the invention, the agent is an antisense oligonucleotide. Preferably, the antisense oligonucleotide is a single-stranded DNA.

In the case of an antisense nucleic acid that is an oligonucleotide, the oligonucleotide may be modified at the base moiety, sugar moiety, or phosphate backbone, and may include other appending groups to facilitate the function of the antisense nucleic acid.

The oligonucleotide may be modified at any position on its structure with constituents generally known in the art. For example, the antisense oligonucleotide may comprise at least one modified base moiety which is selected from the group including 5-fluorouracil, 5-bromouracil, 5-chlorouracil, 5-iodouracil, hypoxanthine, xanthine, 4-acetylcytosine, 5-(carboxyliydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The oligonucleotide may also include at least one modified sugar moiety selected from the group including, but not limited to, arabinose, 2-fluoroarabinose, xylulose, and hexose. In addition, the oligonucleotide may include at least one modified phosphate backbone, such as a phosphorothioate, a phosphorodithioate, a phosphoramidothioate, a phosphoramidate, a phosphordiamidate, a methylphosphonate, an alkyl phosphotriester, and a formacetal or any analogue thereof.

Antisense oligonucleotides according to the present invention may be synthesized by standard methods known in the art. For example, phosphorothioate oligonucleotides may be synthesized by the method as described in Stein et al. (1988) Nucl. Acids Res. 16: 3209.

Alternatively, the antisense nucleic acid may be produced intracellularly by transcription from an exogenous sequence. For example, a vector can be introduced into the cell and an antisense RNA nucleic acid may then be produced by transcription. As will be appreciated, the vector in this case will contain a sequence encoding the antisense nucleic acid and a suitable constitutive or inducible promoter for driving expression of the antisense nucleic acid known in the art. For example, a suitable promoter for driving expression in a large variety of cell types is the CMV promoter.

Such a vector can remain episomal or become chromosomally integrated, as long as it can be transcribed to produce the desired antisense RNA. Vectors can be constructed by recombinant DNA technology methods standard in the art. Vectors can be plasmid, viral, or other vectors known in the art, used for the replication and expression in eukaryotic cells.

An effective amount of the agent is an amount of an agent that is capable of altering the expression and/or function in a cell of a polypeptide based on SEQ ID No. 4. As will be appreciated, an effective amount will depend on the particular properties of both the agent and the cell type of interest. In the case where the agent is an antisense nucleic acid, the amount of antisense nucleic acid that will be effective in the inhibition of translation of the target RNA can be determined by standard techniques known in the art.

The present invention also provides a composition for regulating apoptosis and/or cell proliferation, the composition including a polypeptide including an amino acid sequence selected from the group consisting of:

(a) an amino acid sequence of SEQ ID No. 4;

(b) an amino acid sequence of a biologically active fragment of a polypeptide having an amino acid sequence of SEQ ID No. 4; and

(c) an amino sequence at least 90% identical to an amino acid sequence of SEQ ID No. 4, or a biologically active fragment thereof

The polypeptides in the composition according to this form of the present invention may be produced for example using chemical methods to synthesize the amino acid sequence. For example, peptide synthesis can be performed using various solid-phase techniques (as described in Roberge, J. Y. et al. (1995) Science 269:202-204) and automated synthesis may be achieved, for example, using a peptide synthesizer.

The newly synthesized polypeptide may be substantially purified, for example, by preparative high performance liquid chromatography (as described in Creighton, T. (1983) Proteins, Structures and Molecular Principles, WH Freeman and Co., New York, N.Y.). The composition of the synthetic polypeptide may be confirmed by amino acid analysis or sequencing (e.g. by Edman degradation).

Alternatively, the polypeptides may be expressed in cells or in vitro and subsequently purified. To express the polypeptides, the nucleotide sequences may be inserted into appropriate expression vector, i.e., a vector that contains the necessary elements for the transcription and translation of the inserted coding sequence. Methods for isolation of nucleic acids and cloning are as described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express the polypeptides. For example, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The polypeptide so expressed may be purified by a method known in the art, including methods such ammonium sulphate precipitation, various forms of chromatography, affinity purification, or purification of inclusion bodies.

The composition according to the present invention may also contain a preservative, stabiliser, dispersing agent, pH controller or isotonic agent. The composition may also contain a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener. Further details on techniques for formulation and administration may be found in the latest edition of Remington's Pharmaceutical Sciences (Maack Publishing Co., Easton, Pa.)

The composition according to the present invention will be useful for regulating apoptosis and/or cell proliferation in a subject. In this regard, the apoptosis may be any form of apoptosis that occurs in response to a genetic or external response, or be any form of dysregulated apoptosis.

In this regard, examples of diseases or conditions mediated by a dysregulation of apoptosis include viral diseases such as AIDS; fulminant hepatitis; neurodegenerative diseases such as Alzheimer's disease, Parkinson's disease; dementia; demyelination diseases; cancers in which dysregulation of apoptosis is associated with development of the cancerous phenotype; amyotrophic lateral sclerosis; retinitis pigmentosa; cerebellar degeneration; myelodysplasis such as aplastic anemia; ischemic diseases such as myocardial infarction and stroke; hepatic diseases such as alcoholic hepatitis, hepatitis B and hepatitis C; joint diseases such as osteoarthritis; and atherosclerosis.

Examples of cancers in which dysregulation of apoptosis is associated with development of the cancerous phenotype include bladder cancer, bone cancer, brain tumours, breast cancer, cervical cancer, colorectal cancer including cancer of the colon, rectum, anus, and appendix, cancer of the esophagus, Hodgkin's disease, kidney cancer, cancer of the larynx, leukemia, liver cancer, lung cancer, lymphoma, melanoma, moles and dysplastic nevi, multiple myeloma, muscular cancer, non-Hodgkin's lymphoma, oral cancer, ovarian cancer, cancer of the pancreas, prostate cancer, skin cancer, stomach cancer, testicular cancer, teratoma, thyroid cancer, cancer of the uterus.

The compositions utilized in the present invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Compositions for oral administration can be formulated using pharmaceutically acceptable carriers well known in the art in dosages suitable for oral administration. Such carriers enable the compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The compositions of the present invention may be manufactured in a manner that is known in the art, e.g., by means of conventional mixing, dissolving, granulating, dragee-making, levigating, emulsifying, encapsulating, entrapping, or lyophilizing processes.

The composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions suitable for use in the invention include compositions wherein the active ingredients are contained in an effective amount to achieve the intended purpose. The determination of an effective amount will depend upon the particular subject, the properties of the polypeptide and factors such as half life of the polypeptide and transit times.

An effective amount can be estimated initially either in cell culture assays or in animal models, usually mice, rabbits, dogs, or pigs. The animal model may also be used to determine the appropriate concentration range and route of administration. Such information can then be used to determine useful doses and routes for administration in animals and humans.

The present invention also provides an antibody raised against a polypeptide including an amino acid sequence selected from the group consisting of:

(a) an amino acid sequence of SEQ ID No. 4;

(b) an amino acid sequence encoding a biologically active fragment of a polypeptide having an amino acid sequence of SEQ ID No. 4;

(c) an amino sequence at least 90% identical to an amino acid sequence of SEQ ID No. 4, or a biologically active fragment thereof; and

(d) an amino acid sequence encoding an immunogenic fragment of a polypeptide having an amino acid sequence of SEQ ID No. 4.

Antibodies to polypeptides based on SEQ ID No. 4 may be generated using methods that are known in the art. Such antibodies include, polyclonal, monoclonal, chimeric, single chain, Fab fragments, and fragments produced by a Fab expression library.

For the production of antibodies, various hosts including goats, rabbits, rats, mice, humans, and others, may be immunized by injection with the polypeptide or any fragment or oligopeptide thereof that has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol.

Monoclonal antibodies may be prepared using any technique which provides for the production of antibody molecules by continuous cell lines in culture. These include, but are not limited to, the hybridoma technique, the human B-cell hybridoma technique, and the EBV-hybridoma technique (essentially as described in Kohler, G. et al. (1975) Nature 256:495-497; Kozbor, D. et al. (1985) J. Immunol. Methods 81:31-42; Cote, R. J. et al. (1983) Proc. Nat. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (as described in Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454).

Antibody fragments which contain specific binding sites for polypeptides based on SEQ ID No. 4 may also be generated. For example, such fragments include F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (as described in Huse, W. D. et al. (1989) Science 254:1275-1281).

Various immunoassays may be used for screening to identify antibodies having the desired specificity. Numerous protocols for competitive binding or immunoradiometric assays using either polyclonal or monoclonal antibodies with established specificities are known in the art.

Antibodies so produced are useful for detecting the presence of a polypeptide based on SEQ ID No. 4 expressed in a cell. Various methods are known in the art for using antibodies to detect proteins, as described in Sambrook, J, Fritsch, E. F. and Maniatis, T. Molecular Cloning: A Laboratory Manual 2nd. ed. Cold Spring Harbor Laboratory Press, New York. (1989). The presence of such polypeptides may indicate the apoptotic or proliferative status of the cell.

Accordingly, in another form, the present invention also provides an antibody that detects a polypeptide including an amino acid sequence selected from the group consisting of:

(a) an amino acid sequence of SEQ ID No. 4;

(b) an amino acid sequence encoding a biologically active fragment of a polypeptide having an amino acid sequence of SEQ ID No. 4;

(c) an amino acid sequence at least 90% identical to an amino acid sequence of SEQ ID No. 4, or a biologically active fragment thereof; and

(d) an amino acid sequence encoding an immunogenic fragment of a polypeptide having an amino acid sequence of SEQ ID No. 4.

The present invention also provides a detectably labelled nucleotide probe, the probe including a nucleotide sequence that hybridizes under stringent conditions to a nucleic acid sequence having a nucleotide sequence of SEQ ID No. 3 or a nucleic acid having a nucleotide sequence complementary to SEQ ID No. 3.

The detectably labelled nucleotide probe may any single-stranded RNA or DNA probe that includes a nucleotide sequence that is capable of hybridising under stringent conditions to any part of the nucleotide sequence of SEQ ID No. 3 or the nucleotide sequence complementary to SEQ ID No. 3.

Preferably, the detectably labelled probe includes a nucleotide sequence that is identical to at least 15 contiguous nucleotides of SEQ ID No. 3 or a sequence complementary to SEQ ID No. 3.

Absolute complementarity, although preferred, is not required, as long as the detectably labelled probe is able to hybridize under stringent conditions to the target nucleic acid. As will be appreciated, the ability to hybridize will depend on both the degree of complementarity and the length of the probe. Methods known in the art may be used to formulate possible probes.

The detectable label utilised in the probe may be any suitable label known in the art. For example, the probe may be labelled with radionucleotides such as ³²P or ³⁵S, and the hybridisation of the probe detected by methods such as autoradiography or phosphorimaging.

Types of nonradioactive labeling and detection systems used include the biotin-streptavidin and the digoxigenin-antidigoxigenin system. In the biotin system biotin is incorporated in the probe by using biotinylated dNTPs during probe synthesis. The incorporated biotin is detected directly by avidin or streptavidin or an anti-biotin antibody conjugated to a fluorochrome or an enzyme such as alkaline phosphatase or horseradish peroxidase. The digoxigenin-anti-digoxigenin system uses digoxigenin (DIG), a cardenolide steroid isolated from Digitalis plants.

In the case of oligonucleotide probes, the probe may be synthesized by chemical synthesis by a method known in the art. For larger RNA probes, the probes may be prepared by transcription of an appropriate template essentially as described in Sambrook, J, Fritsch, E. F. and Maniatis, T. Molecular Cloning: A Laboratory Manual 2nd. ed. Cold Spring Harbor Laboratory Press, New York. (1989).

For larger DNA probes, the DNA may be prepared for example from a plasmid template followed by nick translation or oligo-labelling of an appropriate DNA fragment, essentially as described in Sambrook, J, Fritsch, E. F. and Maniatis, T. Molecular Cloning: A Laboratory Manual 2nd. ed. Cold Spring Harbor Laboratory Press, New York. (1989). As will be appreciated, a double stranded DNA probe will need to be denatured for hybridisation.

For example, a double-stranded labelled probe may be prepared by nick translation of a DNA fragment containing all or part of SEQ ID NO. 3 with ³²P or ³⁵S dNTP, DNAse I, and the Klenow fragment of E.coli polymerase 1. For preparation of a labelled RNA probe, all or part of SEQ ID No. 3 may be cloned into a suitable plasmid vector and the resulting plasmid then linearised with an appropriate restriction enzyme. The linearised plasmid may then serve as a template to produce a labelled anti-sense RNA probe with SP6 RNA polymerase or T7 RNA polymerase using ³²P or ³⁵S NTP.

Stringent hybridisation conditions are conditions that allow complementary nucleic acids to bind to each other within a range from at or near the Tm (Tm is the melting temperature) to about 20° C. below Tm.

For example, conditions for allowing the probe to hybridise under stringent conditions to SEQ ID No. 3 or a sequence complementary to SEQ ID No. 3 are as follows: prehybridization may be performed in a prehybridization solution (eg 6×SSC (1×=150 mM NaCl, 15 mM sodium citrate, pH 7.0), 5× Denhardt's reagent (1 g/l each of Ficoll, Polyvinyl-pyrrolidone, Bovine Serum Albumin), 0.5% SDS, 100 ug/ml denatured, fragmented salmon sperm DNA and 50% formamide) for 2 to 12 hours. Hybridizition of the probe with the target (ie filter) may then be performed under conditions such as 6×SSC, 0.5% SDS, 100 ug/ml denatured, fragmented salmon sperm DNA, 50% formamide at 42° C. or 37° C. overnight. The filter may then be washed with 2×SSC and 0.5% SDS at room temperature for 5 min, 2×SSC and 0.1% SDS at room temperature for 15 min, 0.1×SSC and 0.1% SDS at room temperature and for 30 min at 37 ° C. Finally, the filter may be washed at 68° C. in 0.1×SSC, 0.5 % SDS.

The nucleic acid may be DNA (eg genomic DNA, cDNA or synthetic DNA) or RNA (eg mRNA) to be detected. For example, the nucleic acid may be a mRNA present in a sample derived from a tissue or cell encoding an amino acid sequence of SEQ ID No. 4 or an amino acid sequence at least 90% identical to an amino acid sequence of SEQ ID No. 4.

To allow detection of the nucleic acid, the sample will be prepared by a suitable method known in the art. For example, in the case of detection of mRNA, total RNA may be prepared from cells or a tissue of interest, essentially as described in Sambrook, J, Fritsch, E. F. and Maniatis, T. Molecular Cloning: A Laboratory Manual 2nd. ed. Cold Spring Harbor Laboratory Press, New York. (1989), and reverse transcription then performed.

In another form, the present invention also provides a method of detecting a nucleic acid encoding an amino acid sequence of SEQ ID No. 4 or an amino acid sequence at least 90% identical to an amino acid sequence of SEQ ID No. 4, the method including the step of hybridizing to the nucleic acid a detectably labelled nucleotide probe that hybridizes to the nucleic acid under stringent conditions.

The present invention also provides a method of detecting a nucleic acid encoding an amino acid sequence of SEQ ID No. 4 or an amino acid sequence at least 90% identical to an amino acid sequence of SEQ ID No. 4, the method including the step of hybridizing to the nucleic acid a first nucleic acid that specifically amplifyies the nucleic acid in conjunction with a second nucleic acid.

The nucleic acid to be detected may be DNA (eg genomic DNA, cDNA or synthetic DNA) or RNA (eg mRNA) to be detected.

For example, the nucleic acid to be detected may be a mRNA present in a sample derived from a tissue or cell encoding an amino acid sequence of SEQ ID No. 4 or an amino acid sequence at least 90% identical to an amino acid sequence of SEQ ID No. 4. In this case, the mRNA may be prepared from a tissue or cell by a method known in the art. Alternatively, the nucleic acid may be a genomic DNA.

The first nucleic acid may be any single stranded nucleic acid capable of specifically amplifying the nucleic acid in conjunction with a second nucleic acid. Preferably, the first nucleic acid includes a nucleotide sequence that is identical to at least 15 contiguous nucleotides of SEQ ID No. 3 or a sequence complementary to SEQ ID No. 3.

The second nucleic acid may be any nucleic acid that allows the amplification of a nucleic acid encoding an amino acid sequence of SEQ ID No. 4 or an amino acid sequence at least 90% identical to an amino acid sequence of SEQ ID No. 4, in conjunction with the first nucleic acid.

For example, in the case of the detection of a mRNA, the second nucleic acid may be a nucleic acid that hybridises to a region of a mRNA distinct to the region that the first nucleic acid may hybridise to, such as another part of the mRNA or its polyA tail. Alternatively, the second nucleic acid may hybridize to a sequence attached to the mRNA, such as a nucleotide sequence fused to the 5′-end of the mRNA, as occurs for RACE.

Specific amplification of the nucleic acid may be achieved by a method such as PCR using appropriate conditions. In the case of detection of mRNA, RT-PCR will be performed.

For example, suitable cycling conditions are as follows: 95° C. for 1 min then 56° C. for 30 sec, then 72° C. for 1 min, repeated 30 times. For RT-P CR, the mixture is incubated for 60 min at 37° C. and inactivated at 95° C. for 5 mins.

### DESCRIPTION OF THE PREFERRED EMBODIMENTS

Reference will now be made to experiments that embody the above general principles of the present invention. However, it is to be understood that the following description is not to limit the generality of the above description.

### EXAMPLE 1

Cell Lines

R2 is a rat cerebellum neural cell line as described in Rabizadeh, S. et al. (1993). Science 261: 345-348. The R2P and R2L1 cell lines were constructed as follows:

The human full length cDNA of p75 NTR was obtained by an RT-P CR method using isolated fetal brain mRNA as the template, with the cDNA produced cloned into pUC12 (Amersham BioSciences) for sequencing. The p75NTR cDNA fragment was recovered and ligated into pBluescript 11 KS(−). The plasmid was digested with BamHI and the p75NTR cDNA fragment was cloned into pXT-1 vector BglII site. The correct orientated clone was selected for further use. The purified pXT-1 and pXT1/p75NTR plasmids were transfected into a cell line for packaging retroviral expression vectors, PA317 (Miller, A. D. and Buttimore, C. (1986). Mol. Cell Biol. 6:2895-2902.) by calcium phosphate co-precipitated method. Positive clones were screened by culturing the transfected cells in G418 (400 μg/ml) containing medium. The pseudovirus was collected from the high titering cell. R2P is a pXT-1 transfected cell line. R2L1 is an established p75NTR-expressing R2 cell line that was transfected with a pXT-1/p75NTR expression vector and expresses the human neurotrophin receptor p75NTR and was established by transfecting R2 cells with a retroviral expression vector. Apoptosis of the R2L1 cell line can be induced by serum deprivation at 24h. Apoptosis of the R2P cell line can not be induced by serum deprivation at 48h.

The cells were maintained in DMEM (GIBCO, Grand Island, N.Y.) supplemented with 10% fetal bovine serum, 100 units/ml penicillin, 100 mg/ml streptomycin, and 2 mmol/L L-glutamate.

### EXAMPLE 2

Effect of Neurotrophins and p75NT Antibody on Apoptosis of R2L1 Cell Line

R2P and R2L1 cells were cultured as above. To trigger apoptosis, the culture medium containing 10% FCS was removed and the cells were washed twice with PBS and incubated in the culture medium without 10% FCS. Under serum deprivation, the cells were cultured for a different time in the presence or absence of mouse NGF, recombinant human NT3 or BDNF (a final concentration of 100 ng/ml). To observe the effects of p75^{NTR} on apoptosis, the cells were cultured for different periods of time in the presence of the antibody to p75^{NTR} or normal mouse IgG (500 ng/ml). Apoptosis was analysed by flow cytometric analysis. Triple samples were analysed in each experiment. These experiments were repeated at least twice. During the screening process after transfection, more than 20 clones were identified which expressed p75^{NTR}. All these clones had been characterized to show their expression of p75^{NTR} as determined by Northern blotting and by immunostaining. Further analysis showed that p75^{NTR} DNA was integrated into different host genomes (data not shown). Since all experiments were performed on the R2L1 clone, here we only present the data on this clone. The Northern blotting analysis showed that the human p75^{NTR} was highly expressed in R2L1 cells but not in R2P control vector infected cells.

Immunocytochemical studies showed that R2L1 cells stained with the mouse monoclonal antibody to the human p75^{NTR}, but no staining was seen in the control R2P cells. Stable integration was achieved as evidenced by all daughter cells expressing the transfected gene p75^{NTR} These results indicate that the transfected p75^{NTR} gene can be duplicated along with genomic DNA during mitosis. These results indicate that p75^{NTR} was transcribed and translated in R2L1 cells but not in control R2P cells which were transfected with the PXT-1 blank vector. The expression of p75^{NTR} is very stable and cells which have been maintained in the culture for three years still express a high level of p₇₅NTR

### EXAMPLE 3

Flow Cytometric Analysis of Cell Apoptosis

Cells were washed with PBS twice, suspended in 5ml 70% prechilled ethanol, washed again with PBS twice and resuspended in 1 ml PBS. After digesting RNA for 30 min with 50 U ml RNaseA, the cells were cooled immediately on ice. The cells were stained with 65 μg/ml propidium iodide (PI) in the dark at 4° C. for 1 h. After filtering cells through mesh, the fluorescence of individual cells were measured with a flow cytometer (Becton Dickinson FACS420) essentially as described in Darzynkiewicz et al. (1994). Assays of cell viability: discrimination of cells dying by apoptosis. In: Darzynkiewiez, Z; Crismann, H. A.; Robinson, J. R. (Eds), Methods in Cell Biology: Flow Cytometry. Academic Press, New York pp 1-43.

### EXAMPLE 4

p75^{NTR} Mediates Apoptosis of R2L1 Cells Triggered by the Serum Deprivation

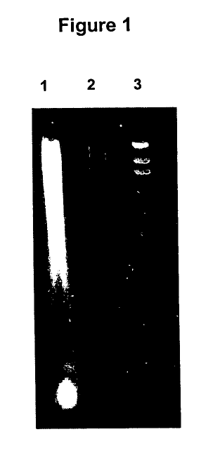

DNA fragmentation and nucleus chromosome DNA degradation is a widely used hallmark for apoptosis. The sub G1 peak (called the apoptotic peak) observed in flow cytometric analysis is also an important quantitative method for detecting apoptosis. Both methods were used in these experiments to identify whether p75^{NTR} expression vector transfected R2L1 cells underwent apoptosis. The results of the DNA fragmentation assay are shown in FIG. 1, which shows significant DNA fragmentation when the R2L1 cell line is deprived of serum containing medium (lane 1) versus R2L1 cultured with serum containing medium after 24 h (lane 2), with equal amounts of DNA loaded. Lane 3 is k DNA cut with HindIII as a marker.

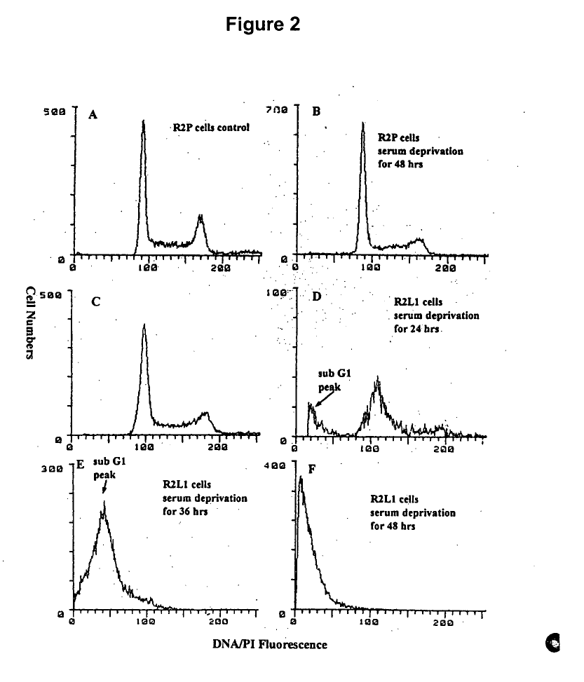

The result of the DNA fragmentation study was further confirmed by the flow cytometric analysis, as shown in FIG. 2. There was no apoptotic peak (sub G1 peak) detected in R2P cells cultured for 24 h in the presence (FIG. 2A) or absence (FIG. 2B) of 10% FCS. After propidium iodide staining, two major fluorescent cell peaks were detected at 90 and 170 nm emission wavelengths, respectively. There was also no apoptotic peak detected in R2L1 cells cultured in the presence of 10% FCS (FIG. 2C). The graph profile of R2L1 cells was similar to that of R2P cells. A significant apoptotic peak (sub G1 pea) was detected in R2L1 cells cultured under the condition of serum deprivation for 24 h (FIG. 2D). The number of normal fluorescent cells was dramatically reduced (see peaks at 90 and 170 nm, FIG. 2D). Thirty-six hours after the removal or serum, a major apoptotic peak of R2L1 cells was observed but no normal fluorescent cell peaks seen (FIG. 2E), indicating all R2L1 cells became apoptotic at this stage. Forty-eight hours after the removal of the serum from the culture, both apoptotic and normal cell peaks disappeared (FIG. 2F), indicating all cells were dead. These results were reproducible in two different experiments.

### EXAMPLE 5

Differential Display of Genes Expressed in Normal Cells and in Cells Undergoing Apoptosis

To induce apoptosis, the culture medium containing 10% FCS was removed and the cells were washed twice with PBS and then incubated in the culture medium without 10% FCS. In the condition of serum deprivation, the cells were cultured for 24-48 h. When R2L1 cell apoptosis was observed, isolated mRNA from R2P and R2L1 was isolated and differential display used to identify genes differentially expressed between cells treated as follows: serum-containing cultured R2L1 cells, serum-depriving cultured R2L1 cells and serum-depriving cultured R2P cells.

The isolation of total was carried out essentially as described in Emmett, M. and Petrack, B. (1988) Anal. Biochem. 174(2):658-661. Reverse transcription was carried out in a 25 μl reaction volume with 2 μg purified total RNA, 1× reverse transcription reaction buffer (50 mM Tris-HCl, 5 mmol/L MgCl₂, 40 mmol/L KCl, pH 8.5), 10 mmol/l DDT, 400 mmol/l dNTPs, 2.5 μmol/l one nucleotide-anchored oligo dT11N ( N is A,G or C) 3′primer, 40 U of RNasin and 200 U M MLV reverse transcriptase. For detecting the quality of reverse transcription, 1 μCi/μl α-32P-dATP was added into an aliquot of 5 μl reaction mixture. All the reaction mixtures were incubated at 37° C. for 60 min, and then inactivated at 95° C. for 5 min.

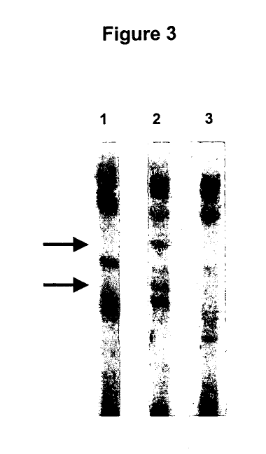

Differential display was performed essentially as described by Liang et al. (1992) Science 257:967-971. In 20 μl P CR reaction volume was added 2 μl reverse transcription reaction mixture, 1×PCR reaction buffer (50 mmol/L KCl, 10 mmol/L Tris-HCl pH 8.3, 1.5 mmol/L MgCl₂), 2.5 μmol/l oligo dT11N, 0.5 μmol/l 5′ random arbitrary primer, 1.25 mmol/l MgCl2, α-³²P-dCTP or α-³³S-dCTP and 0.5 μl Taq DNA polymerase (5 U/ml). The PCR cycling parameters were as follows: 94° C. for 30 sec, 40° C. for 90 sec, 72° C. for 40 sec for total 40 cycles followed by 72° C. for 10 min. The 5′ random arbitrary primers used are CAAGCGAGGT, CAGTGAGCTG and GTCACGGMG. The amplified cDNAs were then separated on a 6% DNA sequencing gel. A number of amplified cDNAs were found to be differentially expressed, as shown in FIG. 3.

Recovery and re-amplification of the differentially expressed cDNAs was achieved by excision of bands from the sequencing gel. The autoradiogram and sequencing gel were oriented with needle punches. After developing the film, differentially expressed cDNA bands were located by either marking with a clean pencil or cutting through the film. The gel slice along with the 3 mm paper was incubated in 100 μl dH₂O for at least 10 minutes. After rehydration of the polyacrylamide gel, the cDNA was recovered by incubating the gel slice for 1 h in a tightly capped microfuge tube. The tube was centrifuged at 15,000g for 10 min, and 4 μl of supernatant was re-amplified in a 50 μl reaction volume using the same primer set and PCR conditions as used in the mRNA display except that dNTP concentrations were at 20 μmol/l and no isotope was added. The PCR samples were run on a 1.5% agrose gel and the cDNAs fragments were recovered by purification using low melting agarose gel.

Recovered cDNA fragments were ligated into the HindIII site of pUC12 vector for sequencing and transformed into E.coli DH5α. Clones were identified by digesting either with BamH I/HindIII or EcoR I/HindIII. Sequencing was carried out using the Taq Trace DNA sequence System kit from Promega Co.

Two DNA fragments, SEQ ID No. 1 and SEQ ID No. 2, were found to differentially expressed and sequenced.

The DNA sequence of SEQ ID No. 1 is as follows:

GGGACAAAACTTTATAACTACAAGCACTTAAGCCTCAAAATTCTTGACTT
TTTCTTTAATGACTATAGTATAACCCTCAGTTGGTCACATGTCTACACAT
TATTTCCAGTTGATAACAAGTAGCGGTGTTTTCCATATGTAATTCAGATC
TGAACTTAATGGCAATAAATGGTTTAAATATTTGCGAAAAAAAAAAA

The DNA sequence of SEQ ID No. 2 is as follows:

CAGGTCACGGAAGCCAGTCATGCCGGAGACACCGGCTTCTGGGAAGCCGC
CCAGGTCTCATTCCTCCCTGCTGTTTGGAGGCAGCATCTCCTCTTTTTAT
GGAGGGCCCGTCCTTTTTTCTTACAAATTCTTCAATAAAGACACATTCTT
GAGGCGAAAAAAAAAAA

Sequence analysis showed that both of these two fragments contain a 3′polyA tail and a 3′polyA tail-adding signal in the 17 nucleotides upstream from 5′-end of the polyA (regularly for eucaryotic mRNAs, the distance is 13-20 nucleotides). After comparison with known sequences in GenBank, these two sequences were found to be novel sequences.

### EXAMPLE 6

The Expression of mRNAs Encoding SEQ ID Nos 1 and 2 is Higher in Apoptotic Cells

To determine the expression level of mRNAs encoding the nucleotide sequences SEQ ID No. 1 and SEQ ID No. 2 in normal and apoptotic cells, Northern analysis of total RNA was performed using the SEQ ID No. 1 and SEQ ID No. 2 clones as probes, as follows:

A labeled probe was obtained by the nick translation using the cloned SEQ ID No. 1 and SEQ ID No. 2 fragments as templates and ³²P dNTP, DNAse I and the Klenow fragment of E.coli DNA polymerase 1.

The total RNAs were isolated from serum-containing cultured R2L1 cells, serum-deprived cultured R2L1 cells and serum-deprived cultured R2P cells. The total RNA was separated according to size by gel eletrophoresis through a denaturing agarose gel and transferred to nitrocellulose. The RNA of interest was located by hybridisation with the radiolabeled DNA probe followed by autoradiography, using the β-Actin as a control to calculate the expression level of SEQ ID Nos 1 and 2 in differential cell lines.

Prehybridization was performed first in Prehybridization Solution: 6×SSC (1×=150 mM NaCl, 15 mM sodium citrate, pH 7.0), 5× Denhardt's reagent (1 g/l each of Ficoll, Polyvinyl-pyrrolidone, Bovine Serum Albumin), 0.5% SDS, 100 μg/ml denatured, fragmented salmon sperm DNA and 50% formamide at 42° C. overnight and then the filter was washed with 2×SSC and 0.5% SDS at room temperature for 5 min. The filter was then washed with 2×SSC and 0.1% SDS at room temperature for 15 min followed by 0.1×SSC and 0.1% SDS at room temperature for 30 min. The amount of radioactivity (as determined by a Geiger counter) then determined if high temperature or low salt solution was to be used again. If used, the temperature was taken to 68° C. and the wash solution was 0.1×SSC, 0.5% SDS.) Quantitation was performed by obtaining the relative ratio of the band intensity of specific fragments over the band density of the internal β-actin controls. A percentage for every fragment relative to the corresponding β-actin from serum-containing R2L1 cells was then made.

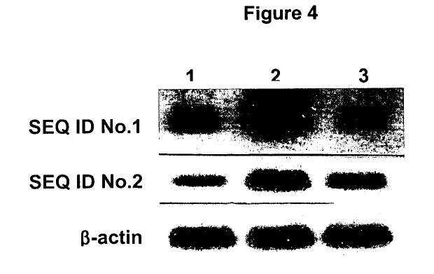

The results are shown in FIG. 4. The expression levels of mRNAs encoding SEQ ID Nos 1 and 2 was found to be higher in apoptotic serum-deprived R2L1 cells than those in normal serum-containing cultured R2L1 cells or serum-deprived R2P cells. Using β-actin as an internal reference to compare relative expression levels, the level of mRNAs encoding SEQ ID No.1 in serum-deprived R2L1 cells was 70% higher than that of serum-containing R2L1 cells and 79% higher than that of serum-deprived R2P cells. The level of mRNAs encoding SEQ ID No. 2 was 76% higher than that of serum-containing R2L1 cells and 79% higher than that of serum-deprived R2P cells higher than these two controls, respectively.

These results demonstrate that mRNAs encoding SEQ ID Nos 1 and 2 are significantly upregulated in cells undergoing apoptosis.

### EXAMPLE 7

SEQ ID NO. 1 Encodes a Novel 109 Amino Acid Protein

To isolate a cDNA encoding the entire coding region of the SEQ ID No. 1 mRNA, rapid amplification of cDNA ends (RACE) was performed on cDNAs isolated from total RNA isolated rat brain mRNA.

RACE was performed using a Marathon-Ready™ cDNA kit purchased from CLONTECH with rat brain mRNA, according to the manufacturer's protocol. Two primers were used: AP1 (5′-CCATCCTMTACGACTCACTATAGGGC3′, which is a universal primer of the Marathon-Ready™ cDNA kit for the inserted cDNA fragments) and a SEQ ID No. 1 specific primer (5′-AGCCGAGAGTTGGTTTGGGGATTC-3′). The amplified PCR fragment was cloned into pGEM-3Zf(+) (Promega) and sequenced to obtain the full length cDNA of the fragment. The nucleotide sequence (SEQ ID. No.3) of the cDNA is shown in FIG. 5.

It was found that the gene has 901 base pairs and encodes a 109 amino acid protein (SEQ ID No. 4) with a PI of 10.4. The protein encoded by the cDNA was designated Apoptosis Related Basic Protein (ARBP).

The extent of sequence homology (using the CLUSTALAW algorithm) of the ARBP protein with the human homologue (GenBank Accession No. NM_{—}016098) and the mouse homologue (GenBank Accession No. NM_{—}018819) is shown in FIG. 6. The ARBP protein has 91% identity and 95% homology with the human homologue, and 99% identity and 99% homology with the mouse homologue.

The extent of identity (using the BLAST algorithm) of the nucleotide sequence of the ARBP nucleic acid with the nucleotide sequence of the human homologue (GenBank Accession No. NM_{—}016098) is shown in FIG. 7. The nucleotide sequence has 89% sequence identity with the human homologue.

The extent of identity (using the BLAST algorithm) of the nucleotide sequence of the ARBP nucleic acid with the nucleotide sequence of the mouse homologue (GenBank Accession No. NM_{—}018819) is shown in FIG. 8. The nucleotide sequence has 90% sequence identity with the mouse homologue.

For comparison, the extent of identity (using the BLAST algorithm) of the nucleotide sequence of the mouse homologue (GenBank Accession No. NM_{—}018819) with the nucleotide sequence of the human homologue (GenBank Accession No. NM_{—}016098) is shown in FIG. 9. The nucleotide sequence has 90% sequence identity with the human homologue.

### EXAMPLE 8

ARBP mRNA is Increased in Lesioned Dorsal Root Ganglia as Compared with Contralateral Dorsal Root Ganglia

Total RNA was prepared from L4 and L5 DRG ganglia (n=3) according to the acid guanidinium thiocyanate-phenol-chloroform method (Chomczynski, P. & Sacchi, N. 1987 Single-step method of RNA isolation by acid guanidinium thiocyanate-phenol-chloroform extraction. Anal. Biochem., 162, 156 159.) using an RNA isolation kit (Advanced Biotechnologies, Leatherhead, UK). The RNA was treated with DNase (Promega, USA) to remove possible contaminating genomic DNA and then directly subjected to first-strand cDNA synthesis. PCR primers for amplification of ARBP and glyceraldehyde-3-phosphate dehydrogenase (GAPDH) control were designed corresponding to the coding region of the genes as follows: Primer I (ARBP cDNA 5′ primer) 5′GGGATCCAACAGCACGGCCATG-3′ and primer 11 (ARBP cDNA 3′ primer) 5′-GGAATTCATTGATMGGCAGCCGAGA-3′; GAPDH primers, sense 5′-TGCTGGTGCTGAGTATGTCG-3′ and antisense 5′-GCATGTCAGATCCACAACGG-3′. The housekeeping gene GAPDH was amplified separately and in parallel to serve as an internal control. PCR reactions were performed in a 30 μL volume using Red-hot DNA polymerase (Advanced Biotechnologies) on a Perkin DNA Thermal Cycler (Perkin Elmer, USA). All PCR samples were heated at 95° C. for 2 min and then amplified in cycles with a programmed profile of 95° C., 30 s; 55° C., 30 s; 72° C., 60 s; followed by a final incubation at 72° C. for 10 min after the last cycle. PCR cycle numbers were 33 for NGF and NT3, and 25 for GAPDH, which were within the determined linear range of standard curves for the individuals.

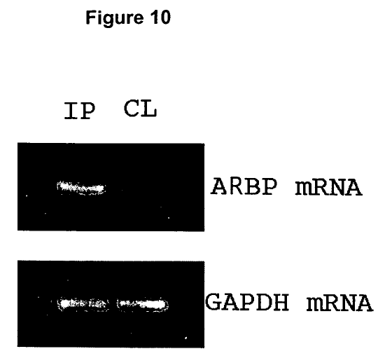

The results are shown in FIG. 10. The increase in mRNA after nerve lesion suggests that this gene may be related to the response of DRG cells to nerve injury. Since transection of sciatic nerve can result in cell death in the DRG, this increase may implicate a role for ARBP in cell death.

### EXAMPLE 9

Detection of ARBP by Western Analysis

Fresh dorsal root ganglia, superior cervical ganglia, trigeminal ganglia and olfactory bulb were dissected from adult rats, and homogenized in Tris buffer with a protease inhibitor tablet (Roche) at ration of 1 mg tissue per 10 microlitres of inhibitor. The homogenate was centrifuged and supernatant was used for SDS-PAGE. 10 microlitres of the sample was loaded with loading buffer and separated by electrophoresis. The protein was transferred to nitrocellulose membrane and probed with a polyclonal antibody against a C-terminal 14 amino acid peptide sequence of ARBP (Gly Arg Leu Ile Asn Tyr Glu Met Ser Lys Arg Pro Ser Ala), prepared as described in Example 10. The bands were visualized with a chemiluminescent method (Roche) on an X-ray film].

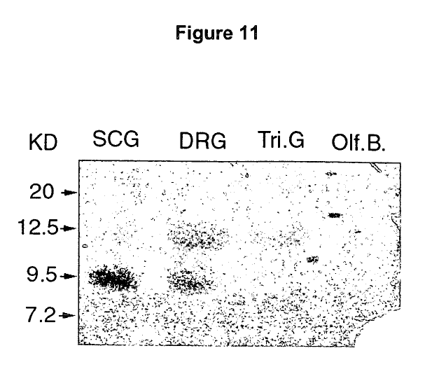

The results are shown in FIG. 11. As can be seen, a band of the predicted size for ARBP(approximately 12 kD) was detected. A smaller band was also detected, which may represent a proteolytic fragment.

### EXAMPLE 10

Immunohistochemical Analysis of ARBP

A chemically synthesized C-terminal 14 amino acids of ARBP Gly Arg Leu lie Asn Tyr Glu Met Ser Lys Arg Pro Ser Ala was prepared and used as an peptide antigen to immunize rabbits. The peptide was conjugated with keyhole limpet hemocyanin (K LH) and was injected into two rabbits. 3 mg of peptide was dissolved in 1 ml PBS and the peptide solution was added into 1 ml of K LH (7 mg/ml). The mixture was added with 2 ml of 0.2% of glutaraldehyde and incubated at room temperature overnight. 10 μl of saturated ammonium chloride was added to the mixture to block unreactive groups of glutaraldehyde. The solution was dialysed against water overnight and was ready for immunization. 400 μg of peptide was diluted in 1 ml PBS and emulsified with 1 ml of complete Feund's adjuvant and injected into a rabbit subcutaneously (s.c). Every two weeks following the first injection, 200 μg of peptide in 1 ml PBS emulsified in incomplete Freund's adjuvant was injected s.c. into the same rabbit. Two months after the first injection, the rabbit was bled after obtaining a high titre antibody to the peptide.

The antibody from the serum was affinity purified on a peptide column. The peptide used for immunization protocol was dissolved in carbonate buffer 0.1 M, pH 9.5. Then 0.5 mg of Cyanogen bromide (CnBr)-activated sepharose (Pharmacia) was added into a small column filled with 0.1 M HCl and the column was rotated for 10 min. The column was washed twice with 5 volumes of HCl and then 5 volumes of sodium carbonate once. The OD280 nm of peptide was measure by a spectrophotometer. The solution was then added into the column whose outlet was blocked. The column was rotated for one hour at room temperature. Then the peptide solution was let out and its OD measured. The incorporation rate was calculated based on OD reading and the rate was >90%. The column was then washed with tris-buffered saline (TBS) and ready for antibody purification. 5 mls of antiserum to ARBP was diluted in TBS 1:1 and filtered and loaded on the column. After dropping out, the column was washed with TBS until the OD of outflow was the same as the inflow. The antibody was eluted with glycine buffer (pH 2.5) and immediately neutralized to pH 7.4 with Tris buffer. The peak with antibody was collected and dialysed against PBS and aliquoted for immunohistochemlstry and for Western blot analysis.

To determine whether the gene is expressed at the protein level, immunohistochemlstry was performed in tissues from adult rats. The purified antibody at 1 μg/ml was used for immunohistochemlstry.

Immunohistochemlstry was performed as follows: normal adult Sprague Dawley rats were anaesthetised and perfused with 4% paraformaldehyde. Different tissues were dissected and sectioned on a cryostat microtome. The sections at 30 micrometer were incubated in 1 μg/ml of affinity purified antibody overnight. After extensive washing in PBS-tween-20, the sections were either directly incubated FITC-labelled donkey anti-rabbit IgG or biotinylated goat anti-rabbit IgG for 1 h at room temperature. In the former condition, the section will be visualized under fluorescent microscope. In the later conditions the section will be further incubated in an ABC kit for an additional hour with washes between these incubations. The sections were developed in diaminobensidine solution with nickel sulphate enhancement.

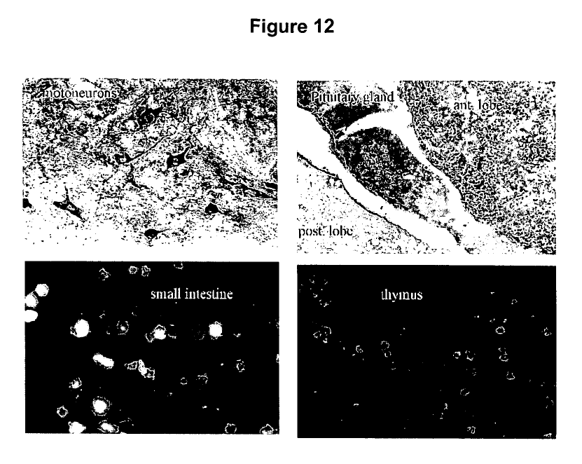

The results are shown in FIG. 12 and demonstrate indicate that ARBP is widely distributed tissues such as intestine, lymph nodes, spleen, liver, thymus, salivary gland. It is also found in neural tissues such as in Schwann cells, motor neurons, substantia nigra, pituitary gland and many non-neuronal cells. These data demonstrate that ARBP is expressed in the cells from a variety of tissues and is not localised to any one particular cell type.

Further studies (data not shown) have also demonstrated that in addition to the above cell types, ARBP is also expressed in the following cells in vivo: cells in the ventral horn (neurons), adrenal medulla (neurons), adrenal cortex, dorsal root ganglion, trigeminal ganglia, hypoglossal nucleus, peripheral nerve (Schwann cells), caudal ventral lateral medulla, cerebellar deep nuclei, singular cortex, dorsal column, dorsal horn, footpad sweat gland, gut, hippocampus, kidney medulla, liver, lung, lymph nodes, mid brain, salivary gland, skin, spleen, substantia nigra, sweat gland, testes, and thymus.

In addition, ARBP expression has also been detected in the following cell lines: C6 (Rat Glial Cell Line), KELLY-(Human Neuroblastoma Cell Line), PC-12-( Rat pheochromocytoma) and SK-N-SH-(Human Neuroblastoma Cell Line)

### EXAMPLE 11

Immunolocalisation of ARBP

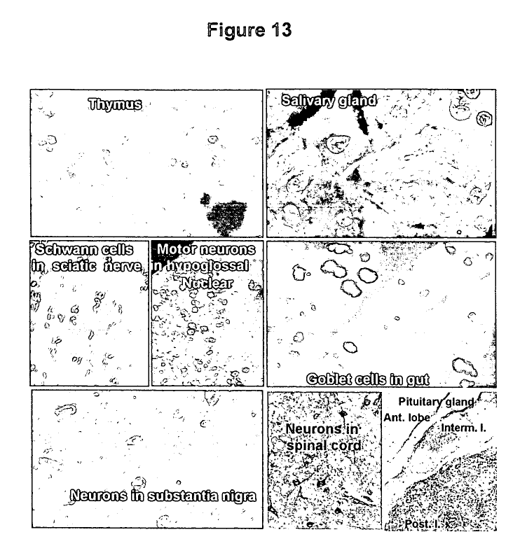

The data is shown in FIG. 13.

ARBP was shown to be localized to a group of cells in the thymus. ARBP is present in cytoplasm. The positive cells showed round shape and scattered in al regions of the thymus. ARBP was also present in the salivary gland and mainly localized to gladular tubules and ducts. ARBP was also found in a variety of neurons including motor neurons in the spinal cord and in the hypoglossal neurons. Significant staining was present in the axons and dendrites of these neurons. ARBP was found in the cytoplasm of Schwann cells in the sciatic nerve and dorsal root ganglia. The most intense labelling of ARBP was found in goblet cells in intestinal tissues. Neuronal cells in substantia nigra was also strongly labelled for ARBP. ARBP was also found in the posterior and intermediate lobes of pituitary gland.

The immunocytochemlstry showed that ARBP is mainly localized in the cell cytoplasm of neurons and other non-neuronal cells but not in the nerve terminals. The staining of ARBP in cytoplasm appeared diffuse but not granular or punctate. These data suggest that ARBP may be a cytoplasmic protein which is not secreted from cell body.

These data further indicate that ARBP is expressed in a large variety of cell lines and may have widespread functions in vivo.

### EXAMPLE 12

Growth of R2L1 Cells is Promoted by Anti-sense Expression of ARBP

To construct sense and antisense ARBP expression vectors, the following primers were used to amplify ARBP DNA for cloning: Primer I (ARBP cDNA 5′ primer) 5′GGGATCCMCAGCACGGCCATG-3′ and primer 11 (ARBP cDNA 3′ primer ) 5′-GGAATTCATTGATAAGGCAGCCGAGA-3′. The primers were used to amplify the coding sequence of ARBP and the PCR products digested with EcoRi and BamHI and cloned into pGEM-3Zf(+). The ARBP coding sequence from the recombinant pGEM-3Zf(+) plasmid was digested with EcoRI, BamHI or EcoRI/XbaI and then cloned into EcoRI, BamHI and EcoRI/XbaI digested pcDNA3.1(+)/Zeo respectively to produce sense and anti-sense ARBP expression vectors.

R2L1 cells were transfected with pcDNA-sense ARBP and pcDNA-antisense ARBP plasmids and were screened by Zeocin (400 μg/ml), and positive clones were named R2L1-ARBPF and R2L1-ARBPR respectively. Transfection was carried out using ProFectionR Mammalian Transfection System from Promega according to the manufacture's protocol. The Zeocin-selected transfected R2L1 cells was designated R2L1-ARBPF and R2L1-ARBPR, respectively, for the sense and antisense expression vectors. The vector pcDNA3.1 (+)/zeo transfecting R2L1 was designated R2L1-pcD, and is a control cell line for cell growth analysis.

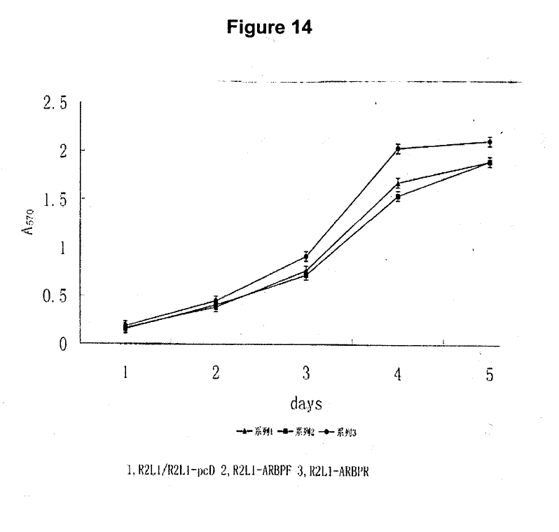

The effect of ARBP expression on the growth of R2L1 cells was determined by the MTT (yellow tetrazolium salt) method. After the cells were trypsinized and counted, 500 cells per well were plated into 96-well plates with 100 μl of complete DMEM medium. Everyday, up to the fifth day, MTT was added to the cultured cells as a final concentration of 1 mg/ml. And then cells were incubated with MTT at 37° C. for 4 hours in a CO₂ incubator, after which time, 100 μl of DMSO was added to dissolve the formed crystals. The results were read using EL instrument (BioRad) at 570 nm. The data is shown in FIG. 14.

The growth curve indicated that the growth of R2L1-ARBPR is faster than the R2L1-ARBPF in culture medium containing serum. Accordingly, the anti-sense expression of ARBP can promote cell proliferation in serum-containing medium. This result indicates that ARBP is normally involved in the regulation of proliferation of cells by suppressing its growth.

These results further suggest ARBP may be involved in cell cycle regulation. Inhibition of ARBP by the anti-sense vector increases the growth of the cells, indicating that ARBP may normally suppress the mitosis of these cells.

### EXAMPLE 13

Anti-sense Expression of ARBP Inhibits Apoptosis

In order to test the effect of ARBP expression on apoptosis, R2L1-ARBPF, R2L1-ARBPR and R2L1-pcD were cultured under conditions of serum deprivation and apoptosis examined by flow cytometry analysis.

Flow Cytometer Analysis of Cell Apoptosis was performed by washing cells (1×10⁶) with PBS twice and suspending cells in 5 ml 70% pre-chilled ethanol by passing through a syringe for 24 h or longer time. The cells were washed with PBS twice and re-suspended in 1 ml PBS. After digesting RNA for half an hour with 50 U/ml RNaseA, the cells were cooled on ice immediately. The cells were stained with 65 μg/ml propidium iodide (PI) in dark box at 4° C. for 1 h. After filtering cell through meshes, the fluorescence of individual cells was measured with flow cytometer (Becton Dickinson FACS420).

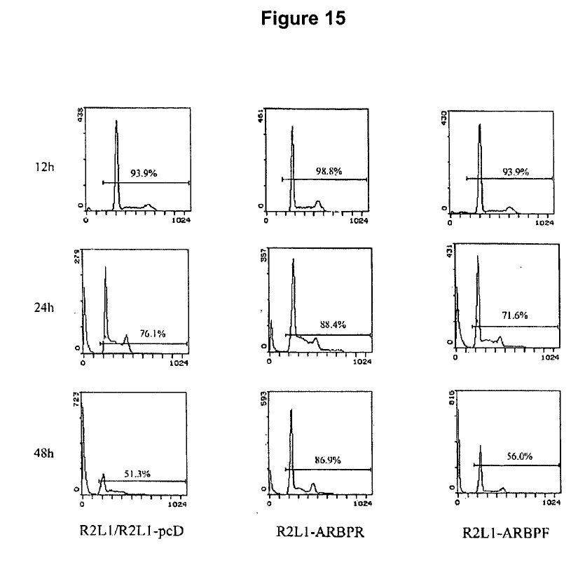

The data is shown in FIG. 15. An apoptotic cell peak was seen in all cells after deprivation of serum for 24 h. The apoptotic cells occupied about 25.7%, 32.2% and 13.2% of total in R2L1-pcD, R2L1-ARBPF and R2L1-ARBPR cells, respectively. There was no statistical significantly difference between the control cells (R2L1-pcD) and ARBP-over-expressed cells (R2L1-ARBPF). However, the suppression of ARBP expression by the anti-sense vector (R2L1-ARBPR) significantly suppressed the apoptosis induced by the serum withdrawal.

This data demonstrates that the anti-sense expression of ARBP can significantly inhibit the apoptosis of R2L1 cells induced by the serum deprivation.

1. A method of regulating apoptosis in a cell, the method including the step of altering the expression and/or function in the cell of a polypeptide including an amino acid sequence selected from the group consisting of:
(a) an amino acid sequence of SEQ ID No. 4;
(b) an amino acid sequence of a biologically active fragment of a polypeptide having an amino acid sequence of SEQ ID No. 4; and
(c) an amino acid sequence at least 90% identical to an amino acid sequence of SEQ ID No. 4, or a biologically active fragment thereof.

2. A method according to claim 1, wherein the cell is a neuronal cell.

3. A method according to claim 1, wherein the cell is a non-neuronal cell.

4. A method according to claim 3, wherein the non-neuronal cell is a non-neuronal cell selected from the group consisting of a non-neuronal cell present in neural and brain tissue, an intestinal cell including a goblet cell, a lymph node cell, a spleen cell, a liver cell, a thymic cell, a salivary gland cell, a pituitary cell, a bladder cell, a bone cell, a breast cell, a cervical cell, a colorectal cell, a kidney cell, a laryngeal cell, a blood cell, a lung cell, a lymphatic cell, a skin cell, a plasma cell, a muscle cell, a cell of the mouth or throat, an ovary, a pancreatic cell, a prostate cell, a stomach cell, a testicular cell, a germ cell, a thyroid cell, and an uterine cell.

5. A method according to claim 1, wherein the apoptosis is mediated by a neurotrophin receptor and/or binding of a neurotrophin to a receptor.

6. A method according to claim 5, wherein the neurotrophin receptor is p75NTR.

7. A method according to claim 1, wherein the apoptosis is associated with a disease or condition involving dysregulation of apoptosis.

8. A method according to claim 7, wherein the disease or condition is selected from the group consisting of viral diseases including AIDS; fulminant hepatitis; neurodegenerative diseases including Alzheimer's disease, Parkinson's disease; dementia; demyelination diseases; cancers in which dysregulation of apoptosis is associated with development of the cancerous phenotype; amyotrophic lateral sclerosis; retinitis pigmentosa; cerebellar degeneration; myelodysplasis including aplastic anemia; ischemic diseases including myocardial infarction and stroke; hepatic diseases including alcoholic hepatitis, hepatitis B and hepatitis C; joint diseases including osteoarthritis; and atherosclerosis.

9. A method of regulating apoptosis in a cell, the method including the step of expressing in the cell a nucleic acid including a nucleotide sequence selected from the group consisting of:
(a) a nucleotide sequence of SEQ ID No. 3, or RNA equivalent thereof;
(b) a nucleotide sequence at least 80% identical to a nucleotide sequence of SEQ ID No. 3, or RNA equivalent thereof;
(c) a nucleotide sequence complementary to SEQ ID No. 3, or RNA equivalent thereof;
(d) a nucleotide sequence at least 80% identical to a nucleotide sequence complementary to SEQ ID No. 3, or RNA equivalent thereof;
(e) a nucleotide sequence encoding a biologically active fragment of a polypeptide having an amino acid sequence of SEQ ID No. 4;
(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and
(g) a nucleotide sequence encoding a ribozyme that cleaves a mRNA encoding an amino acid of SEQ ID No. 4 or an amino acid sequence 90% identical to an amino acid sequence of SEQ ID No. 4.

10. A method according to claim 9, wherein the cell is a neuronal cell.

11. A method according to claim 9, wherein the cell is a non-neuronal cell.

12. A method according to claim 11, wherein the non-neuronal cell is a non-neuronal cell selected from the group consisting of a non-neuronal cell present in neural and brain tissue, an intestinal cell including a goblet cell, a lymph node cell, a spleen cell, a liver cell, a thymic cell, a salivary gland cell, a pituitary cell, a bladder cell, a bone cell, a breast cell, a cervical cell, a colorectal cell, a kidney cell, a laryngeal cell, a blood cell, a lung cell, a lymphatic cell, a skin cell, a plasma cell, a muscle cell, a cell of the mouth or throat, an ovary, a pancreatic cell, a prostate cell, a stomach cell, a testicular cell, a germ cell, a thyroid cell, and an uterine cell.

13. A method according to claim 9, wherein the apoptosis is mediated by a neurotrophin receptor and/or binding of a neurotrophin to a receptor.

14. A method according to claim 13, wherein the neurotrophin receptor is p75NTR.

15. A method according to claim 9, wherein the apoptosis is associated with a disease or condition involving dysregulation of apoptosis.

16. A method according to claim 15, wherein the disease or condition is selected from the group consisting of viral diseases including AIDS; fulminant hepatitis; neurodegenerative diseases including Alzheimer's disease, Parkinson's disease; dementia; demyelination diseases; cancers in which dysregulation of apoptosis is associated with development of the cancerous phenotype; amyotrophic lateral sclerosis; retinitis pigmentosa; cerebellar degeneration; myelodysplasis including aplastic anemia; ischemic diseases including myocardial infarction and stroke; hepatic diseases including alcoholic hepatitis, hepatitis B and hepatitis C; joint diseases including osteoarthritis; and atherosclerosis.

17. A method of regulating proliferation of a cell, the method including the step of altering the expression and/or function in the cell of a polypeptide including an amino acid sequence selected from the group consisting of:
(a) an amino acid sequence of SEQ ID No. 4;
(b) an amino acid sequence of a biologically active fragment of a polypeptide having an amino acid sequence of SEQ ID No. 4 and
(c) an amino acid sequence at least 90% identical to an amino acid sequence of SEQ ID No. 4, or biologically active fragment thereof.

18. A method according to claim 17, wherein the cell is a neuronal cell.

19. A method according to claim 17, wherein the cell is a non-neuronal cell.

20. A method according to claim 19, wherein the non-neuronal cell is a non-neuronal cell selected from the group consisting of a non-neuronal cell present in neural and brain tissue, an intestinal cell including a goblet cell, a lymph node cell, a spleen cell, a liver cell, a thymic cell, a salivary gland cell, a pituitary cell, a bladder cell, a bone cell, a breast cell, a cervical cell, a colorectal cell, a kidney cell, a laryngeal cell, a blood cell, a lung cell, a lymphatic cell, a skin cell, a plasma cell, a muscle cell, a cell of the mouth or throat, an ovary, a pancreatic cell, a prostate cell, a stomach cell, a testicular cell, a germ cell, a thyroid cell, and an uterine cell.

21. A method according to claim 17, wherein the proliferation is mediated by a neurotrophin receptor and/or binding of a neurotrophin to a receptor.

22. A method according to claim 21, wherein the neurotrophin receptor is p75NTR.

23. A method of regulating proliferation of a cell, the method including the step of expressing in the cell a nucleic acid including a nucleotide sequence selected from the group consisting of:
(a) a nucleotide sequence of SEQ ID No. 3, or RNA equivalent thereof;
(b) a nucleotide sequence at least 80% identical to a nucleotide sequence of SEQ ID No. 3, or RNA equivalent thereof;
(c) a nucleotide sequence complementary to SEQ ID No. 3, or RNA equivalent thereof;
(d) a nucleotide sequence at least 80% identical to a nucleotide sequence complementary to SEQ ID No. 3, or RNA equivalent thereof;
(e) a nucleotide sequence encoding a biologically active fragment of a polypeptide having an amino acid sequence of SEQ ID No. 4;
(f) a nucleotide sequence encoding an antisense nucleic acid that reduces the expression in the cell of a polypeptide including an amino acid sequence of SEQ ID No. 4 or an amino acid sequence at least 90% identical to an amino acid sequence of SEQ ID No. 4; and
(g) a nucleotide sequence encoding a ribozyme that cleaves a mRNA encoding an amino acid of SEQ ID No. 4 or an amino acid sequence 90% identical to an amino acid sequence of SEQ ID No. 4.

24. A method according to claim 23, wherein the cell is a neuronal cell.

25. A method according to claim 23, wherein the cell is a non-neuronal cell.

26. A method according to claim 25, wherein the non-neuronal cell is non-neuronal cell selected from the group consisting of a non-neuronal cell present in neural and brain tissue, an intestinal cell including a goblet cell, a lymph node cell, a spleen cell, a liver cell, a thymic cell, a salivary gland cell, a pituitary cell, a bladder cell, a bone cell, a breast cell, a cervical cell, a colorectal cell, a kidney cell, a laryngeal cell, a blood cell, a lung cell, a lymphatic cell, a skin cell, a plasma cell, a muscle cell, a cell of the mouth or throat, an ovary, a pancreatic cell, a prostate cell, a stomach cell, a testicular cell, a germ cell, a thyroid cell, and an uterine cell.

27. A method according to claim 23, wherein the proliferation is mediated by a neurotrophin receptor and/or binding of a neurotrophin to a receptor.

28. A method according to claim 27, wherein the neurotrophin receptor is p75NTR.

29. An agent for regulating cell apoptosis and/or proliferation, wherein the administration of an effective amount of the agent to a cell alters the expression and/or function in the cell of a polypeptide including an amino acid sequence selected from the group consisting of:
(a) an amino acid sequence of SEQ ID No. 4;
(b) an amino acid sequence of a biologically active fragment of a polypeptide having an amino acid sequence of SEQ ID No. 4; and
(c) an amino acid sequence at least 90% identical to an amino acid sequence of SEQ ID No. 4, or a biologically active fragment thereof.

30. An agent according to claim 29, wherein the agent is an antisense nucleic acid.

31. A composition for regulating apoptosis and/or cell proliferation, the composition including a polypeptide including an amino acid sequence selected from the group consisting of:
(a) an amino acid sequence of SEQ ID No. 4;
(b) an amino acid sequence of a biologically active fragment of a polypeptide having an amino acid sequence of SEQ ID No. 4; and
(c) amino acid sequence at least 90% identical to an amino acid sequence of SEQ ID No. 4, or a biologically active fragment thereof.

32. An antibody raised against a polypeptide including an amino acid sequence selected from the group consisting of:
(a) an amino acid sequence of SEQ ID No. 4;
(b) an amino acid sequence encoding a biologically active fragment of a polypeptide having an amino acid sequence of SEQ ID No. 4.
(c) an amino acid sequence at least 90% identical to an amino acid sequence of SEQ ID No. 4, or a biologically active fragment thereof; and
(d) an amino acid sequence encoding an immunogenic fragment of a polypeptide having an amino acid sequence of SEQ ID No. 4.

33. An antibody that detects a polypeptide including an amino acid sequence selected from the group consisting of:
(a) an amino acid sequence of SEQ ID No. 4;
(b) an amino acid sequence encoding a biologically active fragment of a polypeptide having an amino acid sequence of SEQ ID No. 4;
(c) an amino acid sequence at least 90% identical to an amino acid sequence of SEQ ID No. 4, or a biologically active fragment thereof; and
(d) an amino acid sequence encoding an immunogenic fragment of a polypeptide having an amino acid sequence of SEQ ID No. 4.

34. An antisense nucleic acid that reduces the expression in a cell of a polypeptide including an amino acid of SEQ ID NO:4 or an amino acid sequence at least 90% identical to an amino acid sequence of SEQ ID NO. 4
